SEQUENCE LISTING (36 sequences)

SEQ ID NO: 1 - protein, 7 aa, Artificial sequence
  Epitope to which antibody 12A7 binds
WDETKGE

SEQ ID NO: 2 - protein, 7 aa, Artificial sequence
  Epitope to which antibody 4B4 binds
DDRVAIH

SEQ ID NO: 3 - protein, 11 aa, Artificial sequence
  12A7 light chain CDR 1 polypeptide sequence
QNLVQSNGNTY

SEQ ID NO: 4 - DNA, 36 bp, Artificial sequence
  12A7 light chain CDR 1 nucleotide sequence
cagaaccttgtacaaagtaatggaaacacctattta

SEQ ID NO: 5 - protein, 4 aa, Artificial sequence
  12A7 light chain CDR 2 polypeptide sequence
  misc_feature (4)..(4): Xaa can be any naturally occurring amino acid
KVSX

SEQ ID NO: 6 - DNA, 10 bp, Artificial sequence
  12A7 light chain CDR 2 nucleotide sequence
aagtttccaa

SEQ ID NO: 7 - protein, 9 aa, Artificial sequence
  12A7 light chain CDR 3 polypeptide sequence
SQSTRVPYT

SEQ ID NO: 8 - DNA, 27 bp, Artificial sequence
  12A7 light chain CDR 3 nucleotide sequence
tctcaaagtacacgtgttccgtacaca

SEQ ID NO: 9 - protein, 10 aa, Artificial sequence
  12A7 heavy chain CDR 1 polypeptide sequence
GFSLSTSGMG

SEQ ID NO: 10 - DNA, 30 bp, Artificial sequence
  12A7 heavy chain CDR 1 nucleotide sequence
gggttttcactgagcacttctggtatgggt

SEQ ID NO: 11 - protein, 7 aa, Artificial sequence
  12A7 heavy chain CDR 2 polypeptide sequence
IFWDDDK

SEQ ID NO: 12 - DNA, 21 bp, Artificial sequence
  12A7 heavy chain CDR 2 nucleotide sequence
attttctgggatgatgacaag

SEQ ID NO: 13 - protein, 13 aa, Artificial sequence
  12A7 heavy chain CDR 3 polypeptide sequence
ARRSDYNYYSMDY

SEQ ID NO: 14 - DNA, 39 bp, Artificial sequence
  12A7 heavy chain CDR 3 nucleotide sequence
gcgcggcgaagtgactacaattactactctatggactac

SEQ ID NO: 15 - protein, 6 aa, Artificial sequence
  4B4 light chain CDR 1 polypeptide sequence
QDIGSS

SEQ ID NO: 16 - DNA, 18 bp, Artificial sequence
  4B4 light chain CDR 1 nucleotide sequence
caggacattggtagtagc

SEQ ID NO: 17 - protein, 4 aa, Artificial sequence
  4B4 light chain CDR 2 polypeptide sequence
  misc_feature (4)..(4): Xaa can be any naturally occurring amino acid
ATSX

SEQ ID NO: 18 - DNA, 10 bp, Artificial sequence
  4B4 light chain CDR 2 nucleotide sequence
  misc_feature (10)..(10): n is a, c, g, or t
gccacatccn

SEQ ID NO: 19 - protein, 9 aa, Artificial sequence
  4B4 light chain CDR 3 polypeptide sequence
LQYASSPPT

SEQ ID NO: 20 - DNA, 27 bp, Artificial sequence
  4B4 light chain CDR 3 nucleotide sequence
ctacaatatgctagttctcctccgacg

SEQ ID NO: 21 - protein, 8 aa, Artificial sequence
  4B4 heavy chain CDR 1 polypeptide sequence
GFTFSNYA

SEQ ID NO: 22 - DNA, 24 bp, Artificial sequence
  4B4 heavy chain CDR 1 nucleotide sequence
ggattcactttcagtaactatgcc

SEQ ID NO: 23 - protein, 8 aa, Artificial sequence
  4B4 heavy chain CDR 2 polypeptide sequence
ISRGGSYT

SEQ ID NO: 24 - DNA, 24 bp, Artificial sequence
  4B4 heavy chain CDR 2 nucleotide sequence
attagtcgtggtggtagttacacc

SEQ ID NO: 25 - protein, 15 aa, Artificial sequence
  4B4 heavy chain CDR 3 polypeptide sequence
ARHGYNYDDGAWFAN

SEQ ID NO: 26 - DNA, 45 bp, Artificial sequence
  4B4 heavy chain CDR 3 nucleotide sequence
gcaagacatggatataattacgacgacggggcctggtttgctaac

SEQ ID NO: 27 - protein, 113 aa, Artificial sequence
  12A7 full variable light chain sequence (polypeptide)
DIMLTQSPLSLSVTLGDQASISCRSSQNLVQSNGNTYLTWYLQKPGQSPKVLINKVSNRFYGVPDRFSGSGSGTDFTLRISRVEAEDLGIYFCSQSTRVPYTFGGGTKLEIRR

SEQ ID NO: 28 - DNA, 338 bp, Artificial sequence
  12A7 full variable light chain sequence (nucleotide)
gatatcatgctgacccaatctccactctccctgtctgtcactcttggagatcaggcctccatctcttgcagatctagtcagaaccttgtacaaagtaatggaaacacctatttaacttggtacctgcagaagccaggccagtctccaaaggtcctgatcaacaaagtttccaaccgattttatggggtcccagacaggttcagtggcagtggatcagggacagatttcacactcaggatcagcagagtggaggctgaggatctgggaatttatttctgctctcaaagtacacgtgttccgtacacattcggaggggggaccaagctggaaataagacg

SEQ ID NO: 29 - protein, 121 aa, Artificial sequence
  12A7 full variable heavy chain sequence (polypeptide)
QQDLQQSGPGILQPTQTLSLTCSFSGFSLSTSGMGVSWIRQSSNMGLEWLAHIFWDDDKRYNPSLRSRLTLSKDTSSSQVFLMITSVSTADSATYYCARRSDYNYYSMDYWGQGTAVTVSS

SEQ ID NO: 30 - DNA, 363 bp, Artificial sequence
  12A7 full variable heavy chain sequence (nucleotide)
cagcaagatctgcagcagtctggccctgggatattgcagcccacccagaccctcagtctgacttgttctttctctgggttttcactgagcacttctggtatgggtgtgagttggattcgtcaatcttcaaatatgggtctggagtggctggcacacattttctgggatgatgacaagcgctataatccctccctgaggagccgactcacgctctccaaggatacctccagtagccaggtatttctcatgatcaccagtgtgagtactgcagattctgccacatactactgtgcgcggcgaagtgactacaattactactctatggactactggggtcaaggaaccgcagtcaccgtctcctca

SEQ ID NO: 31 - protein, 107 aa, Artificial sequence
  4B4 full variable light chain sequence (polypeptide)
DIMLTQSPSSLSASLGERVSLTCRASQDIGSSLNWLQQEPDGTIKRLIYATSSLDSGVPKRFSGSRSGSDYSLTISSLESEDFVDYYCLQYASSPPTFGGGTKLEIK

SEQ ID NO: 32 - DNA, 322 bp, Artificial sequence
  4B4 full variable light chain sequence (nucleotide)
gatatcatgctgacccaatctccatcctccttatctgcctctctgggagaaagagtcagtctcacttgtcgggcaagtcaggacattggtagtagcttaaactggcttcaacaggaaccagatggaactattaaacgcctaatctacgccacatccagtttagattctggtgtccccaaaaggttcagtggcagtaggtctgggtcagattattctctcaccatcagcagccttgagtctgaagattttgtagactattactgtctacaatatgctagttctcctccgacgttcggtggaggcaccaagctggaaatcaaac

SEQ ID NO: 33 - protein, 121 aa, Artificial sequence
  4B4 full variable heavy chain sequence (polypeptide)
VKLQESGGGLVKPGGSLKLSCAASGFTFSNYAMSWVRQNPEKRLEWVATISRGGSYTYYPDSVKGRFTISRDNAKNTLYLQMNSLRSEDTAMYFCARHGYNYDDGAWFANWGQGTLVTVSA

SEQ ID NO: 34 - DNA, 366 bp, Artificial sequence
  4B4 full variable heavy chain sequence (nucleotide)
taggtgaaactgcaggagtctgggggaggcttagtgaagcctggagggtccctgaaactctcctgtgcagcctctggattcactttcagtaactatgccatgtcttgggttcgccagaatccggagaagaggctggagtgggtcgcaaccattagtcgtggtggtagttacacctactatccagacagtgtgaagggtcgattcaccatctccagagacaatgccaagaacaccctgtatctgcaaatgaacagtctgaggtctgaggacacggccatgtatttctgtgcaagacatggatataattacgacgacggggcctggtttgctaactggggccaagggactctggtcactgtctctgca

SEQ ID NO: 35 - protein, 734 aa, Homo sapiens
  Mcm5 (polypeptide)
MSGFDDPGIFYSDSFGGDAQADEGQARKSQLQRRFKEFLRQYRVGTDRTGFTFKYRDELKRHYNLGEYWIEVEMEDLASFDEDLADYLYKQPAEHLQLLEEAAKEVADEVTRPRPSGEEVLQDIQVMLKSDASPSSIRSLKSDMMSHLVKIPGIIIAASAVRAKATRISIQCRSCRNTLTNIAMRPGLEGYALPRKCNTDQAGRPKCPLDPYFIMPDKCKCVDFQTLKLQELPDAVPHGEMPRHMQLYCDRYLCDKVVPGNRVTIMGIYSIKKFGLTTSRGRDRVGVGIRSSYIRVLGIQVDTDGSGRSFAGAVSPQEEEEFRRLAALPNVYEVISKSIAPSIFGGTDMKKAIACLLFGGSRKRLPDGLTRRGDINLLMLGDPGTAKSQLLKFVEKCSPIGVYTSGKGSSAAGLTASVMRDPSSRNFIMEGGAMVLADGGVVCIDEFDKMREDDRVAIHEAMEQQTISIAKAGITTTLNSRCSVLAAANSVFGRWDETKGEDNIDFMPTILSRFDMIFIVKDEHNEERDVMLAKHVITLHVSALTQTQAVEGEIDLAKLKKFIAYCRVKCGPRLSAEAAEKLKNRYIIMRSGARQHERDSDRRSSIPITVRQLEAIVRIAEALSKMKLQPFATEADVEEALRLFQVSTLDAALSGTLSGVEGFTSQEDQEMLSRIEKQLKRRFAIGSQVSEHSIIKDFTKQKYPEHAIHKVLQLMLRRGEIQHRMQRKVLYRLK

SEQ ID NO: 36 - DNA, 2534 bp, Homo sapiens
  Mcm5 (mRNA)
ggaaaaccagaggcgcagtcatgtcgggattcgacgatcctggcattttctacagcgacagcttcgggggcgacgcccaggccgacgaggggcaggcccgcaaatcgcagctgcagaggcgcttcaaggagttcctgcggcggtaccgagtgggcaccgaccgcacgggcttcaccttcaaatacagggatgaactcaagcggcattacaacctgggggagtactggattgaggtggagatggaggatctggccagctttgatgaggacctggccgactacttgtacaagcagccagccgagcacctgcagctgctggaggaagctgccaaggaggtagctgatgaggtgacccggccccggccttctggggaggaggtgctccaggacatccaggtcatgctcaagtcggacgccagcccttccagcattcgtagcctgaagtcggacatgatgtcacacctggtgaagatccctggcatcatcatcgcggcctctgcggtccgtgccaaggccacccgcatctctatccagtgccgcagctgccgcaacaccctcaccaacattgccatgcgccctggcctcgagggctatgccctgcccaggaagtgcaacacagatcaggctgggcgccccaaatgcccattggacccgtacttcatcatgcccgacaaatgcaaatgcgtggacttccagaccctgaagctgcaggagctgcctgatgcagtcccccacggggagatgcccagacacatgcagctctactgcgacaggtacctgtgtgacaaggtcgtccctgggaacagggttaccatcatgggcatctactccatcaagaagtttggcctgactaccagcaggggccgtgacagggtgggcgtgggcatccgaagctcctacatccgtgtcctgggcatccaggtggacacagatggctctggccgcagctttgctggggccgtgagcccccaggaggaggaggagttccgtcgcctggctgccctcccaaatgtctatgaggtcatctccaagagcatcgccccctccatctttgggggcacagacatgaagaaggccattgcctgcctgctctttgggggctcccgaaagaggctccctgatggacttactcgccgaggagacatcaacctgctgatgctaggggaccctgggacagccaagtcccagcttctgaagtttgtggagaagtgttctcccattggggtatacacgtctgggaaaggcagcagcgcagctggactgacagcctcggtgatgagggacccttcgtcccggaatttcatcatggagggcggagccatggtcctggccgatggtggggtcgtctgtattgacgagtttgacaagatgcgagaagatgaccgtgtggcaatccacgaagccatggagcagcagaccatctctatcgccaaggctgggatcaccaccaccctgaactcccgctgctccgtcctggctgctgccaactcagtgttcggccgctgggatgagacgaagggggaggacaacattgacttcatgcccaccatcttgtcgcgcttcgacatgatcttcatcgtcaaggatgagcacaatgaggagagggatgtgatgctggccaagcatgtcatcactctgcacgtgagcgcactgacacagacacaggctgtggagggcgagattgacctggccaagctgaagaagtttattgcctactgccgagtgaagtgtggcccccggctgtcagcagaggctgcagagaaactgaagaaccgctacatcatcatgcggagcggggcccgtcagcacgagagggacagtgaccgccgctccagcatccccatcactgtgcggcagctggaggccattgtgcgcatcgcggaagccctcagcaagatgaagctgcagcccttcgccacagaggcagatgtggaggaggccctgcggctcttccaagtgtccacgttggatgctgccttgtccggtaccctgtcaggggtggagggcttcaccagccaggaggaccaggagatgctgagccgcatcgagaagcagctcaagcgccgctttgccattggctcccaggtgtctgagcacagcatcatcaaggacttcaccaagcagaaatacccggagcacgccatccacaaggtgctgcagctcatgctgcggcgcggcgagatccagcatcgcatgcagcgcaaggttctctaccgcctcaagtgagtcgcgccgcctcactggactcatggactcgcccacgcctcgcccctcctgccgctgcctgccattgacaatgttgctgggacctctgcctccccactgcagccctcgaacttcccaggcaccctcctttctgccccagaggaaggagctgtagtgtcctgctgcctctgggcgcccgcctctagcgcggttctgggaagtgtgcttttggcatccgttaataataaagccacggtgtgttcaggtaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2022/0283162 A1
(43) Pub. Date: Sep. 8, 2022

(54) MCM5 ELISA FOR THE DETECTION OF RECURRENT BLADDER CANCER

(71) Applicant: ARQUER DIAGNOSTICS LIMITED, Wearfield, Sunderland (GB)

(72) Inventors: Jacqueline STOCKLEY, Wearfield, Sunderland (GB); Cheryl NYBERG, Wearfield, Sunderland (GB); Ashleigh KENNEDY, Wearfield, Sunderland (GB)

(21) Appl. No.: 17/634,579

(22) PCT Filed: Sep. 1, 2020
(86) PCT No.: PCT/GB2020/052095

§ 371 Date: Feb. 11, 2022

(30) Foreign Application Priority Data
Sep. 2, 2019 (GB) 1912605.1

(51) Int. Cl. G01N 33/574

(52) U.S. Cl. CPC G01N 33/57407; G01N 33/57484; G01N 2474/10; G01N 2458/10

(57) ABSTRACT

The present invention relates to methods, uses, a first MCM5 binding agent and/or a second MCM5 binding agents and kits for detecting the presence or absence of a cancer in a subject and/or diagnosing a subject with a cancer and/or monitoring a subject for recurrence of a cancer.

### FIELD OF THE INVENTION

The present invention relates to methods, uses, a first MCM5 binding agent and/or a second MCM5 binding agents and kits for detecting the presence or absence of a cancer in a subject and/or diagnosing a subject with a cancer and/or monitoring a subject for recurrence of a cancer. In particular, the present invention relates to methods for detecting the presence or absence of a recurrent bladder cancer in a subject and/or diagnosing a subject with a recurrent bladder cancer and/or monitoring a subject for recurrence of a bladder cancer. The present invention also concerns the use of a first MCM5 binding agent and/or a second MCM5 binding agent for detecting the presence or absence of a cancer, e.g. a recurrent and/or bladder cancer, in a subject. The present invention further relates to use of a first MCM5 binding agent and/or a second MCM5 binding agent for monitoring a subject for recurrence of a cancer, e.g. a bladder cancer. The present invention further relates to kits comprising (i) a first MCM5 binding agent; (ii) a second MCM5 binding agent; and/or (iii) a lysis buffer capable of releasing MCM5 from cells in a urine sample; and (iv) instructions for use of the first MCM5 binding agent, the second MCM5 binding agent, and/or the lysis buffer.

### BACKGROUND

Cancer recurrence is a significant medical challenge. One such cancer associated with high recurrence rates is bladder cancer, which is the tenth most common type cancer worldwide, accounting for 3% of all new cases. 50% of those diagnosed with bladder cancer go on to die. Notably, bladder cancer has the highest recurrence rate of any known cancer, up to 80%.

Urine cytology is used in combination with cystoscopy for surveillance in the follow up of non-muscle invasive bladder cancer. However, the diagnostic accuracy of urine cytology is low and varies greatly due to the subjective nature of the technique. In addition, cystoscopy requires the insertion of a flexible camera through the urethra into the bladder and can therefore be uncomfortable for the patient undergoing the test as well as requiring a trained medical professional to perform the procedure. Thus, there is an unmet medical need for non-invasive techniques allowing the accurate detection of recurrent cancers and, in particular recurrent bladder cancers.

MCM5 is a known cancer biomarker. However, MCM5 has not been shown to be a useful biomarker for the detection of recurrent bladder cancer.

### SUMMARY OF THE INVENTION

The findings described in the Examples demonstrate that non-invasive measurement of MCM5 in patient urine samples allows accurate the detection of recurrent bladder cancer tumours in the follow up of bladder cancer. In particular, it has been found that assessment of MCM5 in patient samples provides highly sensitive detection of any bladder cancer that is not a non-invasive papillary carcinoma e.g. any high grade bladder cancer or any bladder cancer of stage T1 and above. The inventors compared the performance of a urine-based MCM5 assay to cytology which, in combination with cystoscopy, is considered the gold standard for the surveillance in the follow up of bladder cancer. Surprisingly, it was found that an assay based on the assessment of MCM5 in patient urine samples is vastly superior to cytology in terms of its capacity to accurately detect recurrent bladder cancer. Specifically, the MCM5 assay was significantly more sensitive and had a greater negative predictive value (NPV) than cytological analysis. Based on these findings it is proposed that a urine-based MCM5 assay should replace cytology in the follow-up of recurrent cancer, and in particular recurrent bladder cancer.

The present invention thus provides a method for detecting the presence or absence of a recurrent bladder cancer in a subject, the method comprising:
providing a urine sample previously isolated from the subject;
performing an assay to assess the amount of MCM5 in the sample;
comparing the amount of MCM5 in the sample to a reference;
determining the presence or absence of the recurrent bladder cancer in the subject based on the amount of MCM5 in the sample;

wherein the method does not comprise cytological analysis of the subject or a sample obtained from the subject.

The present invention also provides a method for diagnosing a subject with a recurrent bladder cancer, the method comprising:
providing a urine sample previously isolated from the subject;
performing an assay to assess the amount of MCM5 in the sample;
comparing the amount of MCM5 in the sample to a reference;
diagnosing the subject with a recurrent bladder cancer if the amount of MCM5 measured in the sample is higher than the reference or diagnosing the subject as not having a recurrent bladder cancer if the amount of MCM5 measured in the sample is lower than the reference;

wherein the method does not comprise cytological analysis of the subject or a sample obtained from the subject.

The present invention further provides a method for monitoring a subject for recurrence of a bladder cancer, the method comprising:
(a) providing a urine sample previously isolated from the subject;
(b) performing an assay to assess the amount of MCM5 in the sample;
(c) comparing the amount of MCM5 in the sample to a reference;
(d) determining whether the subject does or does not have a recurrent bladder cancer based on the amount of MCM5 in the sample;
(e) repeating steps (a)-(d), optionally wherein steps (a)-(d) are repeated every month or are repeated once every 2, 3, 4, 5, 6, 7, 8, 9, 10, 11, 12, 18, 24, or 36 months;

wherein the method does not comprise cytological analysis of the subject or a sample obtained from the subject.

Also provided by the present invention is the use of a first MCM5 binding agent and/or a second MCM5 binding agent for detecting the presence or absence of a recurrent bladder cancer in a subject.

Further provided by the present invention is the use of a first MCM5 binding agent and/or a second MCM5 binding agent for monitoring a subject for recurrence of a bladder cancer.

The present invention also provides a first MCM5 binding agent and/or a second MCM5 binding agent for use in a method of diagnosing a subject with a recurrent bladder cancer. The method in which the first MCM5 binding agent and/or the second MCM5 binding agent is used may be a method of the present invention.

Further provided by the present invention is a kit comprising:
(i) a first MCM5 binding agent;
(ii) a second MCM5 binding agent; and/or
(iii) a lysis buffer capable of releasing MCM5 from cells in a urine sample; and
(iv) instructions for use of the first MCM5 binding agent, the second MCM5 binding agent, and/or the lysis buffer in: a method of detecting the presence or absence of a recurrent bladder cancer in a subject, a method for diagnosing a subject with a recurrent bladder cancer, and/or a method of monitoring a subject for recurrence of a bladder cancer.

The present invention may also be applied to other cancer types. Accordingly, for every aspect and embodiment of the present invention disclosed herein which refers to “a recurrent bladder cancer” the present invention also encompasses a corresponding aspect or embodiment which refers to “a cancer” and a corresponding aspect or embodiment which refers to “a recurrent cancer”.

The present invention thus provides a method for detecting the presence or absence of a cancer in a subject, the method comprising:
providing a urine sample previously isolated from the subject;
performing an assay to assess the amount of MCM5 in the sample;
comparing the amount of MCM5 in the sample to a reference;
determining the presence or absence of the cancer in the subject based on the amount of MCM5 in the sample;

wherein the method does not comprise cytological analysis of the subject or a sample obtained from the subject.

The present invention also provides a method for diagnosing a subject with a cancer, the method comprising:
providing a urine sample previously isolated from the subject;
performing an assay to assess the amount of MCM5 in the sample;
comparing the amount of MCM5 in the sample to a reference;
diagnosing the subject with a cancer if the amount of MCM5 measured in the sample is higher than the reference or diagnosing the subject as not having a cancer if the amount of MCM5 measured in the sample is lower than the reference;

wherein the method does not comprise cytological analysis of the subject or a sample obtained from the subject.

The present invention further provides a method for monitoring a subject for recurrence of a cancer, the method comprising:
(a) providing a urine sample previously isolated from the subject;
(b) performing an assay to assess the amount of MCM5 in the sample;
(c) comparing the amount of MCM5 in the sample to a reference;
(d) determining whether the subject does or does not have a cancer based on the amount of MCM5 in the sample;
(e) repeating steps (a)-(d), optionally wherein steps (a)-(d) are repeated every month or are repeated once every 2, 3, 4, 5, 6, 7, 8, 9, 10, 11, 12, 18, 24, or 36 months;

wherein the method does not comprise cytological analysis of the subject or a sample obtained from the subject.

Also provided by the present invention is the use of a first MCM5 binding agent and/or a second MCM5 binding agent for detecting the presence or absence of a cancer in a subject.

Further provided by the present invention is the use of a first MCM5 binding agent and/or a second MCM5 binding agent for monitoring a subject for recurrence of a cancer.

The present invention also provides a first MCM5 binding agent and/or a second MCM5 binding agent for use in a method of diagnosing a subject with a cancer. The method in which the first MCM5 binding agent and/or the second MCM5 binding agent is used may be a method of the present invention.

Further provided by the present invention is a kit comprising:
(i) a first MCM5 binding agent;
(ii) a second MCM5 binding agent; and/or
(iii) a lysis buffer capable of releasing MCM5 from cells in a urine sample; and
(iv) instructions for use of the first MCM5 binding agent, the second MCM5 binding agent, and/or the lysis buffer in: a method of detecting the presence or absence of a cancer in a subject, a method for diagnosing a subject with a cancer, and/or a method of monitoring a subject for recurrence of a bladder cancer.

The present invention thus provides a method for detecting the presence or absence of a recurrent cancer in a subject, the method comprising:
providing a urine sample previously isolated from the subject;
performing an assay to assess the amount of MCM5 in the sample;
comparing the amount of MCM5 in the sample to a reference;
determining the presence or absence of the recurrent cancer in the subject based on the amount of MCM5 in the sample;

wherein the method does not comprise cytological analysis of the subject or a sample obtained from the subject.

The present invention also provides a method for diagnosing a subject with a recurrent cancer, the method comprising:
providing a urine sample previously isolated from the subject;
performing an assay to assess the amount of MCM5 in the sample;
comparing the amount of MCM5 in the sample to a reference;
diagnosing the subject with a recurrent cancer if the amount of MCM5 measured in the sample is higher than the reference or diagnosing the subject as not having a recurrent cancer if the amount of MCM5 measured in the sample is lower than the reference;

wherein the method does not comprise cytological analysis of the subject or a sample obtained from the subject.

Also provided by the present invention is the use of a first MCM5 binding agent and/or a second MCM5 binding agent for detecting the presence or absence of a recurrent cancer in a subject.

The present invention also provides a first MCM5 binding agent and/or a second MCM5 binding agent for use in a method of diagnosing a subject with a recurrent cancer. The method in which the first MCM5 binding agent and/or the second MCM5 binding agent is used may be a method of the present invention.

Further provided by the present invention is a kit comprising:
(i) a first MCM5 binding agent;
(ii) a second MCM5 binding agent; and/or
(iii) a lysis buffer capable of releasing MCM5 from cells in a urine sample; and
(iv) instructions for use of the first MCM5 binding agent, the second MCM5 binding agent, and/or the lysis buffer in: a method of detecting the presence or absence of a recurrent cancer in a subject, a method for diagnosing a subject with a recurrent cancer, and/or a method of monitoring a subject for recurrence of a cancer.

### BRIEF DESCRIPTION OF THE DRAWINGS

FIG. 1 shows graphs presenting the comparisons of diagnostic accuracy of the MCM5 ELISA with cytology for detection of: (A) all types of recurrent bladder cancer; and (B) all non-low grade pTa tumours (i.e. any high grade tumour, or any tumour of stage pT1 and above).

### DETAILED DESCRIPTION

### Definitions

The term “comprises” (comprise, comprising) should be understood to have its normal meaning in the art, i.e. that the stated feature or group of features is included, but that the term does not exclude any other stated feature or group of features from also being present. For example, a lysis buffer comprising a detergent may contain other components.

The term “consists of” should also be understood to have its normal meaning in the art, i.e. that the stated feature or group of features is included, to the exclusion of further features. For example a lysis buffer consisting of a detergent contains detergent and no other components. A lysis buffer comprising a detergent consisting of polysorbate 80 may comprise components other than detergents but the only detergent in the lysis buffer is polysorbate 80.

For every embodiment in which “comprises” or “comprising” is used, we anticipate a further embodiment in which “consists of” or “consisting of” is used. Thus, every disclosure of “comprises” should be considered to be a disclosure of “consists of”.

Cancer

The present invention concerns the detection, diagnosis, and monitoring of cancer, such as recurrent cancer, e.g. recurrent bladder cancer. As used herein, the term “bladder cancer” encompasses any cancer of the bladder, including cancers of the bladder lining (e.g. the urothelium), bladder wall muscle layer (muscularis propria), bladder wall connective tissue layer (lamina propria), the renal pelvis, the ureters, and the urethra.

As used herein, the term “recurrent” refers to a cancer that has returned in the subject after the subject was treated for a primary cancer, e.g. if the subject has recurrent bladder cancer, the subject was previously diagnosed with a primary bladder cancer. The term “recurrent” as used herein encompasses cancers that have recurred: locally i.e. the recurrent cancer is located at the same site, or close to the site, of the primary cancer; or regionally i.e. the recurrent cancer has spread into the lymph nodes or tissues near the site of the primary cancer. Thus, the recurrent bladder cancer may be a locally recurrent bladder cancer or a regionally recurrent bladder cancer. The recurrent bladder cancer may have returned after a period of remission, i.e. a period in which a cancer was absent or undetectable in the subject. The period of remission may be at least 1, 2, 3, 4, 5, 6, 7, 8, 9, 10, 11, 12, 18, 24, or 36 months. The period of remission may be at least one, two, three, four, five, six, seven, eight, nine, or ten years.

The bladder cancer may be non-invasive, for example a non-invasive papillary carcinoma or a carcinoma in situ (CIS). The bladder cancer may be non-muscle-invasive (also referred to as a superficial bladder cancer). Typically, a non-muscle-invasive bladder cancer is one that has only grown into the connective tissue layer of the bladder wall (the lamina propria) but not into the muscle layer of the bladder wall (muscularis propria). The bladder cancer may be muscle-invasive. A muscle-invasive bladder cancer is one that has grown into the muscle layer of the bladder wall (muscularis propria) and sometimes into the fatty layers or surrounding tissue outside the bladder.

The grade of a bladder cancer indicates how abnormal the cells of the bladder cancer appear and therefore how aggressive the bladder cancer is. Bladder cancer cells that appear more abnormal grow more quickly. Bladder cancer grade can be divided into low (cells look relatively normal and are therefore less aggressive) or high (cells are abnormal or poorly differentiated and therefore more aggressive) or can be scored from 1 (less aggressive, slowly growing) to 3 (aggressive, faster growing). A low grade bladder cancer may recur but is less likely to recur than a high grade bladder cancer. A high grade bladder cancer is more likely to recur and grow.

A bladder cancer may also be classified i.e. staged using the Tumour-Node-Metastasis (TNM) staging system. Using the TNM system, the “T′ plus a letter and/or number (0 to 4) is used to describe the size and location of the bladder cancer tumour. For example, the following classes may be used: TX—the primary bladder cancer cannot be evaluated; T0—there is no evidence of a primary bladder cancer in the bladder; Ta—refers to a non-invasive papillary carcinoma; Tis—the bladder cancer is a carcinoma in situ (CIS) or a “flat tumour”; T1—the bladder cancer has spread to the connective tissue layer of the bladder wall (the lamina propria) but not the muscle layer of the bladder wall (muscularis propria); T2—the bladder cancer has spread to the muscle of the bladder wall; T2a—the bladder cancer has spread to the inner half of the muscle of the bladder wall (also referred to as the superficial muscle); T2b—the bladder cancer has spread to the deep muscle of the bladder (the outer half of the muscle); T3—the bladder cancer has grown into the fatty tissue that surrounds the bladder (perivesical tissue); T3a—the bladder cancer has grown into the perivesical tissue, as seen through a microscope; T3b—the bladder cancer has grown into the perivesical tissue and can be observed macroscopically; T4—the bladder cancer has spread to any of the following: the abdominal wall, the pelvic wall, the prostate or the seminal vesicle (in a male), or the uterus or vagina (in a female); T4a—the tumour has spread to the prostate, seminal vesicles, uterus, or vagina; T4b—the tumour has spread to the pelvic wall or the abdominal wall.

Under the TNM staging system, “N” plus a number indicates the extent to which the bladder cancer has spread into the lymph nodes. Lymph nodes near located near to the bladder within the pelvis are termed regional lymph nodes while lymph nodes in other parts of the body are called distant lymph nodes. For example, the following classes may be used: NX—the regional lymph nodes cannot be evaluated; N0—the bladder cancer has not spread to the regional lymph nodes; N1—the bladder cancer has spread to a single regional lymph node in the pelvis; N2—the bladder cancer has spread to two or more regional lymph nodes in the pelvis; N3— the bladder cancer has spread to the common iliac lymph nodes.

Under the TNM staging system, “M” plus a number indicates the extent to which the bladder cancer has spread to other parts of the body i.e. distant metastasis. For example, the following classes may be used: M0—the bladder cancer has not metastasized; M1—there is distant metastasis; M1a—the bladder cancer has spread only to lymph nodes outside of the pelvis; M1b—the cancer has spread other parts of the body.

The TNM classifications may also be grouped as follows.

Stage 0a (Ta, N0, M0): This is an early bladder cancer that is only found on the surface of the inner lining of the bladder. This type of bladder cancer has not invaded the muscle or connective tissue of the bladder wall. This type of bladder cancer is also called non-invasive papillary urothelial carcinoma (Ta, N0, M0).

Stage 0is (Tis, N0, M0): This stage of bladder cancer, also known as a flat tumour or carcinoma in situ (CIS), is found only on the inner lining of the bladder. It has not grown in toward the hollow part of the bladder, and it has not spread to the thick layer of muscle or connective tissue of the bladder (Tis, N0, M0). CIS/Tis is considered a high-grade cancer.

Stage I (T1, N0, M0): The bladder cancer has grown through the inner lining of the bladder and into the lamina propria. The bladder cancer has not spread to the thick layer of muscle in the bladder wall or to lymph nodes or other organs.

Stage II (T2, N0, M0): The bladder cancer has spread into the thick muscle wall of the bladder. It is also called invasive cancer or muscle-invasive cancer. The bladder cancer has not spread to the fatty tissue surrounding the bladder and has not spread to the lymph nodes or other organs.

Stage III: The bladder cancer has spread throughout the muscle wall to the fatty layer of tissue surrounding the bladder (perivesical tissue) or to the prostate in a man or the uterus and vagina in a woman. Alternatively, the cancer has spread to the regional lymph nodes. Stage IIIA: The bladder cancer has grown into the perivesical tissue or has spread to the prostate, uterus, or vagina, but has not spread to the lymph nodes or other organs (T3a, T3b, or T4a; N0; M0). Alternatively, the bladder cancer has spread to a single regional lymph node (T1 to T4a, N1, M0). Stage IIIB: The bladder cancer has spread to two or more regional lymph nodes or to the common iliac lymph nodes (T1 to T4a, N2 or N3, M0).

Stage IV. The bladder cancer has spread into the pelvic wall or abdominal wall, or the bladder cancer has spread to lymph nodes outside of the pelvis or to other parts of the body. Stage IVA: The bladder cancer has spread to the pelvic wall or the abdominal wall but not to other parts of the body (T4b, any N, M0), or the bladder cancer has spread to lymph nodes located outside of the pelvis (any T, any N, M1a). Stage IVB: The bladder cancer has spread other parts of the body (any T, any N, M1b).

The methods and uses of the present invention are useful for monitoring a subject for, diagnosing a subject with, and/or detecting, any recurrent bladder cancer, regardless of stage or grade. Thus, the bladder cancer may be a high grade (HG) bladder cancer, a low grade (LG) bladder cancer, a low grade non-invasive papillary carcinoma (LGpTa), a high grade non-invasive papillary carcinoma (HGTa), a carcinoma in situ (CIS; Tis); a bladder cancer that has invaded the connective tissue layer (lamina propria) but not the muscle layer (muscularis propria) of the bladder wall (i.e. a T1 bladder cancer).

The methods and uses of the present invention have particularly high sensitivity and NPV for detection of bladder cancers which are not low grade non-invasive papillary adenocarcinoma (LGpTa). Thus, in one embodiment, the bladder cancer may be a high grade (HG) bladder cancer, a high grade non-invasive papillary carcinoma (HGTa), a carcinoma in situ (CIS; Tis), a bladder cancer that has invaded the connective tissue layer (lamina propria) but not the muscle layer (muscularis propria) of the bladder wall i.e. a T1 bladder cancer, a T2 bladder cancer, a T3 bladder cancer, a T3a bladder cancer, a T3b bladder cancer, a T4 bladder cancer, a T4 bladder cancer, a T4a bladder cancer, a T4b bladder cancer, or a bladder cancer having the stage 0 is, I, II, III, IIIA, IIIB, IV, IVA, or IVB.

Cytology and Cystoscopy

The present inventors have unexpectedly found that an assay based on the detection of MCM5 in a sample is significantly more sensitive and has a significantly higher NPV for the detection and/or diagnosis of recurrent bladder as compared to cytological analysis. As such, the methods and uses of the present invention are able to accurately detect recurrent bladder cancer without the requirement to perform cytological analysis of the subject or a sample obtained from the subject. Accordingly, the methods and uses of the present invention do not comprise cytological analysis of the subject or a sample obtained from the subject. This should be understood to mean that the methods and uses of the present invention do not require cytological analysis to be performed in order to achieve their stated purpose. Thus, although cytological analysis may be performed on the subject or a sample obtained from the subject for other reasons, cytological analysis of the subject is not required to carry the methods or uses of the present invention. For example, where the method is for detecting the presence or absence of a recurrent bladder cancer in a subject, the presence or absence of the recurrent bladder cancer is determined without performing cytological analysis on the subject or a sample obtained from the subject. Similarly, where the method is for diagnosing a subject with a recurrent bladder cancer, the subject is diagnosed as having, or not having, a recurrent bladder cancer without performing cytological analysis on the subject or a sample obtained from the subject. Further, where the method is for monitoring a subject for recurrence of a bladder cancer, whether the subject does or does not have a recurrent bladder cancer is determined without performing cytological analysis on the subject or a sample obtained from the subject.

As used herein, the term “cytological analysis” refers to a technique in which the morphology (shape and appearance) of cells isolated from a subject are examined under the microscope. As used herein, the term “cytological analysis” encompasses the study of healthy cells, also known as “cytology”, as well as diseased cells, also known as “cytopathology”. Cells having an abnormal morphology can be identified as pre-cancerous or cancerous based on cytological analysis performed by a trained person. As used herein, the term “cytological analysis” may also be understood to encompass any procedure performed on the subject in order to isolate cells for the purpose of assessing the morphology of said cells. For example, cells can be isolated from the subject by fine needle aspiration (FNA) or by brushing or scraping cells from an organ or tissue. Alternatively, cells from may be isolated from a bodily fluid of the subject, such as urine, sputum (phlegm), spinal fluid, pleural fluid, pericardial fluid, or peritoneal fluid.

Urine cytology, in combination with cystoscopy, is considered the gold standard for surveillance in the follow up of non-muscle invasive bladder cancer. However, the diagnostic accuracy of urine cytology is low and varies greatly due to the subjective nature of the technique even when performed by a trained professional. Since the methods and uses of the present invention are performed without cytological analysis and have been shown to be more sensitive and have a higher NPV than this technique, they represent an improvement over the gold standard.

As used herein, the term “cystoscopy” refers to a procedure in which a trained medical practitioner examiners the interior of the bladder of the subject by inserting a cystoscope—a thin, flexible camera—through the urethra and into the bladder. In some embodiments of the methods and uses of the present invention, the subject is “undergoing cystoscopic surveillance”. This can be understood to mean that the subject has undergone at least one cystoscopy procedure on the same day on which the method or use is carried out. Alternatively, the term “undergoing cystoscopic surveillance” can be understood to mean that the subject has undergone at least one cystoscopy procedure within: the preceding one, two, three, four, five, six, or seven days; or the preceding one, two, three or four weeks; or the preceding 1, 2, 3, 4, 5, 6, 7, 8, 9, 10, 11, or 12 months. The term “undergoing cystoscopic surveillance” may also be understood to mean that subject will undergo a cystoscopy procedure within: the next one, two, three, four, five, six, or seven days; or the next one, two, three, or four weeks; or the next 1, 2, 3, 4, 5, 6, 7, 8, 9, 10, 11, or 12 months.

Urine Sample

The methods and uses described herein are applied to a urine sample. A urine sample can be obtained by using any technique known in the art. For example, a urine sample may be obtained from a subject by asking the subject to urinate into a collection vessel, such as a sterile container. As used herein, the term “urine sample” encompasses any sample derived from urine originally obtained from the subject. For example, the urine sample may be processed by centrifugation to obtain e.g. a pellet comprising urinary sediment comprising cells. The supernatant may be discarded and the urinary sediment cell pellet re-suspended in a suitable buffer, such as a lysis buffer according to the invention or otherwise described herein. The urine sample may be processed by passage through a filter in order to collect cells suspended in the urine on said filter.

Subject

The present methods and uses relate to detecting the presence or absence of a recurrent bladder cancer in a subject and/or diagnosing a subject with a recurrent bladder cancer and/or monitoring a subject for recurrence of a bladder cancer. In some embodiments, the subject is a human. In some embodiments, the subject is male. In some embodiments, the subject is female.

In some embodiments, the subject is under the age of 18, 17, 16, 15, 14, 13, 12, 11, 10, 9, 8, 7, 6, 5, 4, 3, 2, or 1 year(s). In some embodiments, the subject is an adult. In some embodiments, the subject is at least 16, 17, 18, 19, 20, 21, 22, 23, 24, or 25 years in age. In some embodiments, the subject is elderly, for example at least 65, 70, 75, 80, 85, or 90 years in age.

In some embodiments, the subject is suffering from a bladder cancer. In some embodiments, the subject is in remission from a bladder cancer. In some embodiments, the subject is suspected of having a bladder cancer. In some embodiments, the subject is healthy. In some embodiments, the subject is at risk of developing a bladder cancer. In some embodiments, the subject has an increased risk of developing a bladder cancer. The subject may be at risk or have an increased risk of developing a bladder cancer where the subject has been previously identified as carrying a genetic marker indicating an increased risk of developing a bladder cancer. The subject may be at risk or have an increased risk of developing a bladder cancer where the subject has a family history of bladder cancer. The subject may be at risk or have an increased risk of developing a bladder cancer where the subject is suffering from, or has previously been diagnosed with a cancer, for example, a non-bladder cancer.

In other embodiments, the subject is a non-human mammal, for example the subject may be a primate, feline, canine, bovine, equine, murine, ovine, or porcine subject.

The subject may be in remission from a bladder cancer e.g. the subject may have been previously diagnosed with a primary bladder cancer which has been treated. The primary cancer may no longer be detectable.

The subject may be asymptomatic of a bladder cancer. For example, an asymptomatic subject may not present with one or more symptoms associated with bladder cancer. Thus, in some embodiments, the subject does not present with haematuria (blood or blood clots in the urine), pain or a burning sensation during urination, frequent urination, feeling the need to urinate many times throughout the night, feeling the need to urinate but not being able to pass urine, and/or lower back pain confined to one side of the body.

Measurement of MCM5

The methods and uses described herein may involve “performing an assay to assess the amount of MCM5 in the sample”. As used herein, the term “assess the amount of MCM5” encompasses both qualitatively and quantitatively evaluating the amount of MCM5. Thus, the methods and uses of the present invention may be carried out by performing an assay which qualitatively assesses the presence or absence of MCM5 in the sample. In such methods and uses, the user does not need to determine a numerical value for the amount of MCM5 in the sample. Alternatively, the methods and uses of the present invention may be carried out by performing an assay which quantitatively assesses the amount of MCM5 in the sample e.g. the concentration of MCM5 in the sample. In such methods and uses, the user may determine a numerical value for the amount of MCM5 in the sample.

The skilled person may use any suitable assay known in the art to assess the amount of MCM5 in the sample. For example, the amount of MCM5 may be measured by interaction with a ligand or ligands, 1-D or 2-D gel-based analysis systems, liquid chromatography, combined liquid chromatography and any mass spectrometry techniques including MSMS, ICAT (R) or iTRAQ (R), agglutination tests, thin-layer chromatography, NMR spectroscopy, sandwich immunoassays, enzyme linked immunosorbent assays (ELISAs), radioimmunoassays (RAI), enzyme immunoassays (EIA), lateral flow/immunochromatographic strip tests, Western Blotting, immunoprecipitation, particle-based immunoassays including using gold, silver, or latex particles and magnetic particles or Q-dots, or any other suitable technique known in the art.

In some embodiments, the assay performed to assess the amount of MCM5 in the sample is an ELISA assay. The ELISA assay may be a sandwich ELISA assay. A sandwich ELISA assay comprises steps of capturing MCM5 to be detected or whose concentration is to be determined using a “capture antibody” already bound to a plate, and detecting how much of MCM5 has been captured using a “detection antibody”. The detection antibody may be pre-conjugated to a label such as the enzyme HRP (Horse Radish Peroxidase). The ELISA plate may then be exposed to the labelled detection antibody, such that the labelled detection antibody binds to the captured at least one biomarker. After exposure to the labelled detection antibody, the ELISA plate should then be washed to remove any excess unbound labelled detection antibody. The washed plate can then be exposed to an agent whose properties are changed by the label (of the detection antibody) in a measurable manner. The amount of the detection antibody may then be determined. For example, if the detection antibody is labelled by e.g. conjugation to HRP, the ELISA plate may be exposed to 3,3′,5,5′-tetramethylbenzidine (TMB) substrate. The amount of the detection antibody, and therefore the amount of MCM5 in the sample, may then be determined by evaluation of the colour change corresponding to the conversion of TMB into a coloured product. The amount of coloured product may be evaluated by measuring optical density (OD) e.g. OD at 450 nm and/or using a reference wavelength of 630 nm.

The ELISA assay may be a qualitative ELISA assay. In the context of the present invention, a qualitative ELISA assay is one that is performed without determining a numerical value for the amount of MCM5 in the urine sample and/or without determining, or using a previously determined, numerical value for the amount of MCM5 as a reference. For example, in a qualitative ELISA assay, the amount of a coloured product (e.g. a coloured product produced by an enzyme linked to a detection antibody) may be compared to a reference. The amount of the coloured product is indicative of the amount of MCM5 without the need to determine a numerical value for the amount of MCM5.

### REFERENCE

The methods and uses of the present invention may involve comparing the amount of MCM5 in the sample to a reference. Just as the amount of MCM5 in the sample may be assessed either qualitatively or quantitatively, the amount of MCM5 in the sample may be compared to the reference either qualitatively or quantitatively. When comparing the amount of MCM5 in the sample to the reference qualitatively, the user does not need to determine a numerical value for the amount of MCM5 in the sample, but may merely determine whether the amount of MCM5 in the sample is the same as, or different, to the reference. Alternatively, a numerical value for the amount of MCM5 in the sample may be compared quantitatively to the reference.

In some embodiments, the reference comprises a positive control and/or a negative control. Thus, the amount of MCM5 in the sample may be compared to a positive control and/or a negative control. Preferably, the reference comprises a positive control and negative control. The amount of MCM5 in the sample may therefore be compared to a positive control and a negative control. The positive control and the negative control may be processed in parallel to the sample. For example, where the assay to assess the amount of MCM5 in the sample is an ELISA, the amount of MCM5 in the positive control and in the negative control may be assessed using an ELISA performed in parallel to the ELISA performed to assess the amount of MCM5 in the sample.

Where the reference comprises a positive control and a negative control, the amount of MCM5 in the sample may be compared with the reference to provide a percentage score, using the following calculation:

$$\frac{( MCM {5}_{sample} - MCM {5}_{negative control} )}{MCM {5}_{postive control}} \times 100$$

where MCM5ₛₐₘₚₗₑ, MCM5_{negative control} and MCM5_{positive control} correspond to the amount of MCM5 in the sample, negative control and positive control, respectively.

For example, where the assay to assess the amount of MCM5 in the sample is an ELISA in which the amount of a coloured product is evaluated by measurement of optical density (OD), the following calculation may be used to provide a percentage score:

$$\frac{( {OD}_{sample} - {OD}_{negative control} )}{{OD}_{postive control}} \times 100$$

where ODₛₐₘₚₗₑ, OD_{negative control} and OD_{positive control} correspond to the OD of the sample, negative control and positive control, respectively.

Suitably, the reference may be an amount of MCM5 based on a sample obtained from one or more healthy individuals and/or sample obtained from a person known to have a cancer. A healthy individual is one who is not presently suffering from a bladder cancer. For example, the healthy individual may be one who has never suffered from a cancer e.g. a bladder cancer.

Where the reference comprises a positive control and a negative control, the positive control may comprise an amount of MCM5 which is known to correspond to the presence of a cancer. The negative control may comprise an amount of MCM5 which is known to correspond to the absence of cancer. For example, since healthy individuals do not produce samples comprising detectable MCM5, the negative control may be a buffer, e.g. lysis buffer, which does not comprise MCM5.

Suitably the reference may be matched to the subject being analysed e.g. by gender, by age, by ethnic background or other such criteria which are well known in the art. For example, the reference may suitably be matched to specific patient sub-groups e.g. elderly subjects, or those with a previous relevant history such as a predisposition to bladder cancer.

Suitably the reference may be matched to the sample type being analysed. For example, the amount of MCM5 in the urine sample previously isolated from the subject may vary depending on the type or nature of the sample. Thus, in some embodiments, it is desirable that the sample type used to determine the reference and the urine sample previously isolated from the subject are of the same type.

In some embodiments, the amount of MCM5 in the sample may be compared to a reference which is an amount of MCM5 in a previous sample from the same subject. This can be beneficial in monitoring the recurrence of bladder cancer in the subject, which can in turn be beneficial in monitoring the course and/or effectiveness of a treatment of the subject. The reference amount of MCM5 may have been determined at least 1, 2, 3, 4, 5, 6, 7, 8, 9, 10, 11, 12, 18, 24, 36 or 48 months, or at least 1, 2, 3, 4, 5, 6, 7, 8 or 10 years prior to carrying out the method or use described herein.

Detection and Diagnosis Cancer

Typically, it is not possible for a diagnostic method to predict with 100% accuracy whether or a cancer is present in a subject. Thus, the wording “determining the presence or absence of the recurrent bladder cancer in the subject” can be understood to refer to determining whether a recurrent bladder cancer is likely to be present or absent in a subject. Likewise, the wording “diagnosing the subject with a recurrent bladder cancer” can be understood to refer to determining whether the subject is likely to have a recurrent bladder cancer.

The present invention provides a method for detecting the presence or absence of a recurrent bladder cancer. In such methods, a recurrent bladder cancer may be determined to be present if the amount of MCM5 in the sample is higher than the reference or a recurrent bladder cancer may be determined to be absent if the amount of MCM5 in the sample is lower than the reference. The present invention provides a method for diagnosing a subject with a recurrent bladder cancer. In such methods, the subject may be diagnosed with a recurrent bladder cancer if the amount of MCM5 in the sample is higher than the reference or the subject may be diagnosed as not having a recurrent bladder cancer if the amount of MCM5 in the sample is lower than the reference. The term “higher” encompasses “statistically significantly higher” and the term “lower” encompasses “statistically significantly lower”, for example as determined by a suitable statistical test known in the art.

For example, where the reference is a mean average amount of MCM5, a recurrent bladder cancer may be determined to be present if the amount of MCM5 in the sample is higher than the reference by at least one, at least two, at least three, or at least four standard deviations.

In one embodiment, a recurrent bladder cancer is likely to be present if the amount of MCM5 in the sample is at least 1.05 times, at least 1.10 times, at least 1.15 times, at least 1.2 times, at least 1.3 times, at least 1.4 times, at least 1.5 times, at least 1.6 times, at least 1.7 times, at least 1.8 times, at least 1.9 times, at least 2.0 times, at least 2.1 times, at least 2.2 times, at least 2.3 times, at least 2.5 times, at least 3 times, at least 3.5 times, at least 4 times, at least 5 times, at least 10 times, at least 25 times, at least 50 times, at least 75 times, or at least 100 times higher than the reference.

Where the reference comprises a positive control and a negative control and the amount of MCM5 in the sample is compared with the reference to provide a percentage score using the following calculation:

$$\frac{( MCM {5}_{sample} - MCM {5}_{negative control} )}{MCM {5}_{postive control}} \times 100$$

a percentage score of greater than about 0.9%, about 0.905%, about 0.91%, about 0.915%, about 0.92%, about 0.925%, about 0.93%, about 0.935%, about 0.94%, about 0.945%, about 0.95%, about 0.955%, about 0.96%, about 0.965%, about 0.97%, about 0.975%, about 0.98%, or about 0.985% indicates that the amount of MCM5 in the sample is higher than the reference. A percentage score of less than about 0.9%, about 0.905%, about 0.91%, about 0.915%, about 0.92%, about 0.925%, about 0.93%, about 0.935%, about 0.94%, about 0.945%, about 0.95%, about 0.955%, about 0.96%, about 0.965%, about 0.97%, about 0.975%, about 0.98%, or about 0.985% indicates that the amount of MCM5 in the sample is lower than the reference.

For example, where the assay to assess the amount of MCM5 in the sample is an ELISA in which the amount of a coloured product is evaluated by measurement of optical density (OD) and the following calculation is used to provide a percentage score:

$$\frac{( {OD}_{sample} - {OD}_{negative control} )}{{OD}_{postive control}} \times 100$$

a percentage score of greater than about 0.9%, about 0.905%, about 0.91%, about 0.915%, about 0.92%, about 0.925%, about 0.93%, about 0.935%, about 0.94%, about 0.945%, about 0.95%, about 0.955%, about 0.96%, about 0.965%, about 0.97%, about 0.975%, about 0.98%, or about 0.985% indicates that the amount of MCM5 in the sample is higher than the reference. A percentage score of less than about 0.9%, about 0.905%, about 0.91%, about 0.915%, about 0.92%, about 0.925%, about 0.93%, about 0.935%, about 0.94%, about 0.945%, about 0.95%, about 0.955%, about 0.96%, about 0.965%, about 0.97%, about 0.975%, about 0.98%, or about 0.985% indicates that the amount of MCM5 in the sample is lower than the reference.

Sequence Homology/Identity

Although sequence homology can also be considered in terms of functional similarity (i.e., amino acid residues having similar chemical properties/functions), in the context of the present document it is preferred to express homology in terms of sequence identity.

Sequence comparisons can be conducted by eye or, more usually, with the aid of readily available sequence comparison programs. These publicly and commercially available computer programs can calculate percent homology (such as percent identity) between two or more sequences.

Percent identity may be calculated over contiguous sequences, i.e., one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues (for example less than 50 contiguous amino acids). For comparison over longer sequences, gap scoring is used to produce an optimal alignment to accurately reflect identity levels in related sequences having insertion(s) or deletion(s) relative to one another. A suitable computer program for carrying out such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package, FASTA (Altschul et al., 1990, J. Mol. Biol. 215:403-410) and the GENEWORKS suite of comparison tools.

Typically sequence comparisons are carried out over the length of the reference sequence. For example, if the user wished to determine whether a given sequence is 95% identical to SEQ ID NO: 27, SEQ ID NO: 27 would be the reference sequence. For example, to assess whether a sequence is at least 80% identical to SEQ ID NO: 27 (an example of a reference sequence), the skilled person would carry out an alignment over the length of SEQ ID NO: 27, and identify how many positions in the test sequence were identical to those of SEQ ID NO: 27. If at least 80% of the positions are identical, the test sequence is at least 80% identical to SEQ ID NO: 27. If the sequence is shorter than SEQ ID NO: 27, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

For the purposes of the present invention, the term “fragment” refers to a contiguous portion of a reference sequence. For example, a fragment of SEQ ID NO: 27 of 50 amino acids in length refers to 50 contiguous amino acids of SEQ ID NO: 27.

In the context of the present application, a homologous amino acid sequence is taken to include an amino acid sequence which is at least 40, 50, 60, 70, 80 or 90% identical to the reference sequence. Most suitably a polypeptide having at least 90% sequence identity to the biomarker of interest will be taken as indicative of the presence of that biomarker; more suitably a polypeptide which is 95% or more suitably 98% identical at the amino acid level will be taken to indicate presence of that biomarker. Suitably said comparison is made over at least the length of the polypeptide or fragment which is being assayed to determine the presence or absence of the biomarker of interest. Most suitably the comparison is made across the full length of the polypeptide of interest.

MCM5

MCM proteins 2-7 comprise part of the pre-replication complexes which form on chromatin and which are essential prerequisites, or licensing factors, for subsequent DNA replication. The MCM protein complexes act as replicative helicases and thus are core components of the DNA replication machinery. MCM proteins are upregulated in the transition from the G0 to G1/S phase of the cell cycle and actively participate in cell cycle regulation. The MCM proteins form an annular structure around the chromatin. The human MCM5 gene maps to 22q13.1 and the mature MCM5 protein consists of 734 amino acids (SEQ ID NO: 35; UNIPROT P33992: HUMAN DNA replication licensing factor MCM5). The term “MCM5” refers to a polypeptide of SEQ ID NO: 35, or a polypeptide 85%, 90%, 95%, 98%, or 100% identical to SEQ ID NO: 35.

Statistical Parameters

The methods and uses described herein allow bladder cancer to be detected with high sensitivity and NPV.

In the field of medical diagnostics and as used herein the term “sensitivity” (also referred to as the true positive rate) refers to a measure of the proportion of actual positives that are correctly identified as such. In other words, the sensitivity of a diagnostic test may be expressed as the number of true positives i.e. individuals correctly identified as having a disease as a proportion of all the individuals having the disease in the test population (i.e. the sum of true positive and false negative outcomes). Thus, a high sensitivity diagnostic test is desirable as it rarely misidentifies individuals having the disease. This means that a negative result obtained by a highly sensitive test has a high likelihood of ruling out the disease.

In the field of medical diagnostics and as used herein, the term “specificity” (also referred to as the true negative rate) refers to a measure of the proportion of actual negatives that are correctly identified as such. In other words, the specificity of a diagnostic test may be expressed as the number of true negatives (i.e. healthy individuals correctly identified as not having a disease) as a proportion of all the healthy individuals in the test population (i.e. the sum of true negative and false positive outcomes). Thus, a high specificity diagnostic test is desirable as it rarely misidentifies healthy individuals. This means that a positive result obtained by a highly specific test has a high likelihood of ruling in the disease.

The specificity and/or sensitivity of a method may be determined by performing said method on samples which are known to be positive samples (e.g. samples from patients having bladder cancer) and/or samples which are known to be negative samples (e.g. samples from healthy individuals). The extent to which the method correctly identifies the known positive samples (i.e. the sensitivity/true positive rate of the method) and/or the known negative samples (i.e. the specificity/true negative rate of the method) can thus be determined.

In the field of medical diagnostics, and as used herein, the term “positive predictive value (PPV)” refers to the proportion of all positive outcomes generated by a diagnostic test that are true positive outcomes. Put another way, PPV can be defined as the number of true positive outcomes divided by the sum of true positive outcomes and false positive outcomes.

In the field of medical diagnostics, and as used herein, the term “negative predictive value (NPV)” refers to the proportion of all negatives outcomes generated by a diagnostic test that are true negative outcomes. Put another way, NPV can be defined as the number of true negative outcomes divided by the sum of true negative outcomes and false negative outcomes.

The PPV or NPV of a method may be determined by performing said method on samples which are known to be positive samples (e.g. samples from patients having bladder cancer) and/or samples which are known to be negative samples (e.g. samples from healthy individuals). The NPV can then be calculated as the number of known negative samples divided by the total number of negative test results. The PPV can be calculated as the number of known positive samples divided by the total number of positive test results.

In the field of medical diagnostics, and as used herein, a “Receiver Operating Characteristic (ROC) curve” refers to a plot of true positive rate (sensitivity) against the false positive rate (1—specificity) for all possible cut-off values. In the field of medical diagnostics, and as used herein, a “Youdens index” refers to the cut-off point at which the distance between the ROC curve and the line of chance (45 degree line) is highest. These terms are well known in the art and to the skilled person.

Optionally, the methods and uses of the present invention, or the methods in which the first MCM5 binding agent and/or the second binding agent are provided for use in according to the present invention have a sensitivity of at least about 30%, at least about 35%, at least about 40%, at least about 45%, at least about 50%, at least about 55%, at least about 60%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.5%. In some embodiments, the methods and uses of the present invention, or the methods in which the first MCM5 binding agent and/or the second binding agent are provided for use in according to the present invention have a sensitivity of at least about 40%.

Optionally, the methods and uses of the present invention, or the methods in which the first MCM5 binding agent and/or the second binding agent are provided for use in according to the present invention have an NPV of at least about 80%, at least about 80.5%, at least about 81%, at least about 81.5%, at least about 82%, at least about 82.5%, at least about 83%, at least about 83.5%, at least about 84%, at least about 84.5%, at least about 85%, at least about 85.5%, at least about 86%, at least about 86.5%, at least about 87%, at least about 87.5%, at least about 88%, at least about 88.5%, at least about 89%, at least about 89.5%, at least about 90%, at least about 90.5%, at least about 91%, at least about 91.5%, at least about 92%, at least about 92.5%, at least about 93%, at least about 93.5%, at least about 94%, at least about 94.5%, at least about 95%, at least about 95.5%, at least about 96%, at least about 96.5%, at least about 97%, at least about 97.5%, at least about 98%, at least about 98.5%, at least about 99%, at least about 99.5%. In some embodiments, the methods and uses of the present invention, or the methods in which the first MCM5 binding agent and/or the second binding agent are provided for use in according to the present invention have an NPV of at least about 85%.

Optionally, the methods and uses of the present invention, or the methods in which the first MCM5 binding agent and/or the second binding agent are provided for use in according to the present invention have a sensitivity of at least about 40% and an NPV of at least about 85%.

Optionally, where the present invention relates to a bladder cancer that is not a low grade non-invasive papillary carcinoma (LGpTa) the methods and uses of the present invention, or the methods in which the first MCM5 binding agent and/or the second binding agent are provided for use according to the present invention may have a sensitivity of at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, or at least about 95%. In some embodiments, where the bladder cancer is a not a low grade non-invasive papillary carcinoma (LGpTa) the methods and uses of the present invention, or the methods in which the first MCM5 binding agent and/or the second binding agent are provided for use in according to the present invention may have a sensitivity of at least about 80%.

Optionally, where the present invention relates to a bladder cancer that is not a low grade non-invasive papillary carcinoma (LGpTa) the methods and uses of the present invention, or the methods in which the first MCM5 binding agent and/or the second binding agent are provided for use in according to the present invention may have an NPV of at least about 95%, at least about 95.2%, at least about 95.4%, at least about 95.6%, at least about 95.8%, at least about 96%, at least about 96.2%, at least about 96.4%, at least about 96.6%, at least about 96.8%, at least about 97%, at least about 97.2%, at least about 97.4%, at least about 97.6%, at least about 97.8%, at least about 98%, at least about 98.2%, at least about 98.4%, at least about 98.6%, at least about 98.8%, at least about 99%, at least about 99.2%, or at least about 99.4%. In some embodiments, where the bladder cancer is not a low grade non-invasive papillary carcinoma (LGpTa) the methods and uses of the present invention, or the methods in which the first MCM5 binding agent and/or the second binding agent are provided for use in according to the present invention may have an NPV of at least about 95%.

Optionally, where present invention relates to a bladder cancer that is not a low grade non-invasive papillary carcinoma (LGpTa) the methods and uses of the present invention, or the methods in which the first MCM5 binding agent and/or the second binding agent are provided for use in according to the present invention may have a sensitivity of least about 80% and an NPV of at least about 95%.

Optionally, where the present inventions relate to a high grade (HG) bladder cancer, the methods and uses of the present invention, or the methods in which the first MCM5 binding agent and/or the second binding agent are provided for use in according to the present invention may have a sensitivity of at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, or at least about 95%. In some embodiments, where the bladder cancer is a HG bladder cancer, the methods and uses of the present invention, or the methods in which the first MCM5 binding agent and/or the second binding agent are provided for use in according to the present invention may have a sensitivity of at least about 75%.

Optionally, where the present inventions relate to a carcinoma in situ (CIS; Tis) the methods and uses of the present invention, or the methods in which the first MCM5 binding agent and/or the second binding agent are provided for use in according to the present invention may have a sensitivity of at least about 30%, at least about 40%, at least about 50%, at least about 60%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, or at least about 96%. In some embodiments, where the bladder cancer is a carcinoma in situ (CIS; Tis) the methods and uses of the present invention, or the methods in which the first MCM5 binding agent and/or the second binding agent are provided for use in according to the present invention may have a sensitivity of at least about 70%.

Optionally, where the present invention relates to a T1 bladder cancer the methods and uses of the present invention, or the methods in which the first MCM5 binding agent and/or the second binding agent are provided for use in according to the present invention may have a sensitivity of at least about 35%, at least about 45%, at least about 55%, at least about 65%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%. In some embodiments, where the bladder cancer is a T1 bladder cancer the methods and uses of the present invention, or the methods in which the first MCM5 binding agent and/or the second binding agent are provided for use in according to the present invention may have a sensitivity of at least about 75%.

The present application demonstrates that the presence or absence of a recurrent bladder cancer may be determined more accurately (e.g. with greater sensitivity and/or NPV) compared to cytological analysis. Thus, optionally, the method may have a greater sensitivity and/or NPV than an equivalent method carried out using a cytological analysis.

Exposing the Urine Sample to a Lysis Buffer

Normal epithelial cells in bladder do not express MCM5 as they are fully differentiated and therefore epithelial cells shed into the urine do not contain MCM5. However, in the presence of a tumour, non-differentiated immature proliferating cells which express MCM5 are present at the surface epithelium lining the bladder, and these MCM5-expressing cells can be shed into the urine. Accordingly, the presence of MCM5 in the urine is indicative of a recurrent bladder cancer. Thus, to detect the presence of absence of a recurrent bladder cancer, it is advantageous to be able to release the MCM5 from the epithelial cells in the urine to allow detection using, for example, an antibody based ELISA. For these reasons, the method and uses of described herein may comprise exposing the sample to a lysis buffer capable of releasing MCM5 from cells in the sample in order to release MCM5 from cells in sample.

Optionally, exposing the urine sample to a lysis buffer capable of releasing MCM5 from cells in the urine sample is carried out on a pellet of the cells in the urine sample obtained by centrifuging the urine sample. Optionally, exposing the urine sample to a lysis buffer capable of releasing MCM5 from cells in the urine sample is carried out on a supernatant sample obtained by centrifuging the urine sample. Preferably, the step of treating the urine sample to release MCM5 from cells in the urine sample is carried out before the step of detecting MCM5 or determining the concentration of MCM5.

Optionally, exposing the urine sample to a lysis buffer capable of releasing MCM5 from cells in the urine sample in order to release MCM5 from cells in the sample comprises:
passing the sample through a filter for capturing cells, such that cells are captured in the filter; and
   (i) passing the lysis buffer through the filter, such that the captured cells are exposed to the lysis buffer; and/or
   (ii) incubating the filter in contact with the lysis buffer for a period of time, such that the lysis buffer causes the captured cells to release the MCM5.

Lysis Buffer

Lysis buffers are generally buffers which are able to lyse cells. In addition to releasing one or more biomarkers from cells, the lysis buffer may be compatible with the method used for subsequent analysis. For example, where the analysis method is double-antibody sandwich ELISA, it may be desirable that the lysis buffer does not degrade the capture antibody bound to the surface of the microtitre plate. Lysis buffers generally but not exclusively comprise one or more detergents (also known as surfactants), one or more salts and a buffering agent. In some instances, the concentrations of these components may affect the efficacy of the lysis buffer.

In one embodiment, a buffer can be said to be a “lysis buffer” if it is capable of lysing at least 10%, 20%, 30%, 40%, 50%, 60%, 70%, 80%, 90%, 91%, 92%, 93%, 94%, 95%, 96%, 97%, 98%, 99%, or substantially all of the cells in a urine sample. In one embodiment, a buffer can be said to be a “lysis buffer” if it is capable of lysing at least 10%, 20%, 30%, 40%, 50%, 60%, 70%, 80%, 90%, 91%, 92%, 93%, 94%, 95%, 96%, 97%, 98%, 99%, or substantially all of the cells in a urine sample where the cells in the non-invasive sample are exposed to the lysis buffer for at least 1 minute, 5 minutes, 10 minutes, 20 minutes, 30 minutes, 60 minutes, 2 hours, 6 hours, 12 hours, 24 hours, or overnight. In one embodiment, a buffer can be said to be a “lysis buffer” if it is capable of lysing at least 10%, 20%, 30%, 40%, 50%, 60%, 70%, 80%, 90%, 91%, 92%, 93%, 94%, 95%, 96%, 97%, 98%, 99%, or substantially all of the cells in a urine sample where the cells in the non-invasive sample are exposed to the lysis buffer for at least 1 minute, 5 minutes, 10 minutes, 20 minutes, 30 minutes, 60 minutes, 2 hours, 6 hours, 12 hours, 24 hours, or overnight at 4° C., 20° C., or room temperature.

In one embodiment, the lysis buffer is capable of releasing MCM5, from cells in the urine sample. A lysis buffer will be considered to be “capable of releasing a biomarker from cells in the urine sample” if the amount of the biomarker (such as MCM5) released is greater than 40%, 50%, 60%, 70%, or 80% the amount released when a buffer comprising 25 mM Tris pH 7.6, 150 mM sodium chloride, 1% sodium deoxycholate, 0.1% sodium dodecyl sulphate (SDS), and 1% Triton-X100 is used. The antibodies used in the assay should be antibodies that bind to the biomarker. For example, where the biomarker is MCM5, a first monoclonal antibody and/or second monoclonal antibody that bind to MCM5 could be used. Preferably 12A7 and 4B4 antibodies are used.

Optionally, the lysis buffer does not denature an antibody. This can be useful where detecting MCM5 or determining the concentration of at least one biomarker comprises detecting using an antibody such as by an ELISA assay. If the lysis buffer does not denature an antibody, then it does not need to be removed before the step of detecting MCM5 or determining the concentration of at least one biomarker. A lysis buffer can be considered to not denature an antibody if the activity of the antibody after exposure to the lysis buffer is at least 40%, 50%, 60%, 70%, or 80% the activity of the antibody prior to exposure to the lysis buffer. The activity of the antibody may be tested using an ELISA assay such as that described in Example 1.

In some embodiments, the lysis buffer comprises a detergent (also referred to as a surfactant). In general detergents are compounds that are known to disrupt cell walls. Detergents are amphiphilic having both hydrophobic and hydrophilic regions. Suitable detergents are well known to the person of skill in the art. Examples of detergents that may suitably be used in a lysis buffer according to the present invention include, but are not limited to, Triton X-100, sodium doceyl sulphate (SDS), and sodium deoxycolate, Optionally, the detergent comprises Triton X-100. Optionally, the detergent comprises or consists of sodium deoxycholate. Optionally, the detergent comprises or consists of sodium dodecyl sulphate (SDS). Optionally, the detergent comprises Triton X-100, sodium doceyl sulphate (SDS), and sodium deoxycolate. Optionally, the detergent comprises Triton X-100 at a concentration between 0.01% and 25%, between 0.01% and 10%, between 0.05% and 5%, between 0.1% and 2%, between 0.5% and 2%, between 0.75% and 1.25%, or about 1%. Optionally, the detergent comprises or consists of sodium deoxycholate at a concentration between 0.1% and 20%, between 0.1 and 10%, between 0.1 and 5%, between 0.5% and 5%, between 0.5% and 2.5%, between 0.75% and 2.5%, between 0.75% and 1.25%, or about 1%. Optionally, the detergent comprises or consists of sodium dodecyl sulphate (SDS) at a concentration between 0.001% and 10%, between 0.01% and 5%, between 0.05% and 5%, between 0.01% and 1%, between 0.05% and 1%, between 0.05% and 0.5%, between 0.075% and 0.25%, or about 0.1%. Optionally, the detergent consists of between 0.5% and 2% of Triton X-100, between 0.5% and 2% of sodium deoxycholate, and between 0.05% and 0.5% of sodium dodecyl sulphate (SDS).

In some embodiments, the lysis buffer comprises a buffer component. A buffer component can be considered to be any component which maintains the pH of the lysis buffer at a pH varying by less than 2.0 pH units, 1.5 pH units, or 1.0 pH units. The buffer component may have a pH of between pH 4 and pH 9, between pH 5 and pH 8.5, between pH 6 and pH 8, between pH 6.5 and pH 8, between pH 7 and pH 8, between pH 7.3 and pH 7.9, between pH 7.4 and pH 7.8, between pH 7.5 and pH 7.7, or about pH 7.6; and/or maintains the pH of the lysis buffer at between pH 4 and pH 9, between pH 5 and pH 8.5, between pH 6 and pH 8, between pH 6.5 and pH 8, between pH 7 and pH 8, between pH 7.3 and pH 7.9, between pH 7.4 and pH 7.8, between pH 7.5 and pH 7.7, or about pH 7.6. For example, the buffer component preferably has a pH between pH 7.4 and pH 7.8.

An example of a buffer that may suitably be used in a lysis buffer according to the present invention is Tris. Optionally, the buffer component comprises or consists of Tris. Optionally, the buffer component comprises or consists of Tris at a concentration greater than 1 mM, between 1 mM and 350 mM, between 5 and 200 mM, between 5 and 100 mM, between 5 and 50 mM, between 5 mM and 40 mM, between 5 mM and 35 mM, between 10 mM and 35 mM, between 15 mM and 35 mM, between 15 mM and 30 mM, between 20 mM and 30 mM, or about 25 mM. The buffer component may consist of Tris at a concentration of between 15 mM and 35 mM.

In some embodiments, the lysis buffer comprises a salt. Various salts are well known to the person skilled in the art. In some embodiments, the salt is added to the lysis buffer to provide a particular level of ionic strength. An example of a salt that can be suitably used in a lysis buffer according to the invention is sodium chloride. Optionally, the lysis buffer comprises sodium chloride at a concentration between 10 mM and 350 mM, between 20 mM and 300 mM, between 50 mM and 250 mM, between 100 mM and 250, between 100 mM and 200 mM, between 125 mM and 175 mM, or about 150 mM. Optionally, the lysis buffer comprises sodium chloride at a concentration of between 100 mM and 200 mM.

Optionally, the lysis buffer comprises:
(i) between 1 mM and 100 mM Tris;
(ii) between 50 mM and 300 mM sodium chloride;
(iii) between 0.1 and 5% sodium deoxycholate;
(iv) between 0.01 and 1% sodium dodecyl sulphate; and/or
(v) between 0.1 and 5% Triton-X100.

Optionally, the lysis buffer comprises:
(i) between 10 mM and 40 mM Tris;
(ii) between 100 mM and 200 mM sodium chloride;
(iii) between 0.5 and 2% sodium deoxycholate;
(iv) between 0.05 and 0.5% sodium dodecyl sulphate; and/or
(v) between 0.5 and 2% Triton-X100.

Optionally, the lysis buffer comprises:
(i) about 25 mM Tris;
(ii) about 150 mM sodium chloride;
(iii) about 1% sodium deoxycholate;
(iv) about 0.1% sodium dodecyl sulphate; and/or
(v) about 1% Triton-X100.

MCM5 Binding Agent

Methods of the present invention may comprise exposing the sample to a first MCM5 binding agent and/or a second MCM5 binding agent. The present invention also concerns uses of a first MCM5 binding agent and/or a second MCM5 binding agent.

As used herein, the term “MCM5 binding agent” refers to any molecule capable of binding to an epitope (fragment) of MCM5, for example an epitope having the sequence of SEQ ID NO: 1 or SEQ ID NO: 2. The term “binding agent” encompasses both antibodies and non-antibody binding agents, for example antibody mimetics such as those described in Yu et al. (2017) Annu Rev Anal Chem 10(1):293-320, which is incorporated herein by reference. In some embodiments, the first MCM5 binding agent and/or the second MCM5 binding agent are antibody mimetics. For example, the first MCM5 binding agent is an antibody mimetic which binds to MCM5 and/or the second MCM5 binding agent is an antibody mimetic which binds to MCM5.

Thus, the first MCM5 binding agent and/or second MCM5 binding agent may be selected from the group consisting of antibodies, antibody mimetics, adnectins, affibodies, affilins, affimers, affitins, alphabodies, anticalins, aptamers, armadillo repeat protein-based scaffolds, atrimers, avimers, DARPins, fynomers, knottins, Kunitz domain peptides, monobodies, and nanofitins.

Antibodies Binding to MCM5

In some embodiments, the first MCM5 binding agent and/or the second MCM5 binding agent are antibodies. For example, the first MCM5 binding agent may be a first monoclonal antibody which binds to MCM5 and/or the second MCM5 binding agent may be a second monoclonal antibody which binds to MCM5. Optionally, the first and second monoclonal antibodies bind to different epitopes of MCM5.

The terms “antibody” and “antibodies” can refer to naturally occurring forms or recombinant antibodies such as single-chain antibodies, chimeric antibodies or humanised antibodies. The terms “antibody” and “antibodies” may also be considered to encompass fragments of antibodies that can bind to a target protein, such as an MCM protein like MCM5. Such fragments may include Fab′2, F′(ab)₂, Fv, single chain antibodies or diabodies. The antibodies may be naturally occurring, full length antibodies (rather than fragments). In a further embodiment, the antibodies are not humanised antibodies.

In general, antibodies are formed from two heavy chains and two lights chains. Each heavy chain is made up of heavy chain constant region (CH) and a heavy chain variable region (VH). Similarly each light chain is made up of light chain constant region (CL) and a light chain variable region (VL). The VH and VL regions comprise complementarity defining regions (CDRs). The CDRs are, primarily responsible for specific binding to the target protein.

It is well within the ability of the person skilled in the art to develop an antibody that binds to MCM5. This may be performed by immunising a mammal such as a mouse, rabbit or guinea pig, with MCM5 or a portion thereof. It may be beneficial to include an adjuvant such as Freund's complete adjuvant. The spleen cells of the immunised mammal are removed and fused with myeloma cells to form hybridoma lines which are immortal given appropriate conditions and which secrete antibodies. The hybridomas are separated into single clones and the antibodies secreted by each clone are evaluated for their binding ability to the biomarker protein (e.g. MCM5).

Alternatively, the skilled person may produce recombinant antibodies which bind to MCM5 based on the amino acid and nucleotide sequence information provided herein.

In some embodiments of the methods of the invention, the urine sample is exposed to a first monoclonal antibody which binds to MCM5 and/or a second monoclonal antibody which binds to MCM5. As discussed above, such embodiments may involve performing an ELISA assay or a sandwich ELISA assay which comprises exposing the urine sample to a first monoclonal antibody described herein and/or a second monoclonal antibody described herein. Preferably, the first monoclonal antibody and/or the second monoclonal antibody bind specifically to MCM5. Preferably, the first monoclonal antibody binds to SEQ ID NO: 1. Preferably, the second monoclonal antibody binds to SEQ ID NO: 2.

In some embodiments, the first monoclonal antibody and/or the second monoclonal antibody will bind to an epitope (fragment) of MCM5 (for example SEQ ID NO: 1 or SEQ ID NO: 2). Thus, the term “antibody which binds to MCM5” refers to an antibody that binds to an epitope of MCM5, such as SEQ ID NO: 1 or SEQ ID NO: 2.

For the purposes of the present invention, the term ‘binding affinity’ refers to the ability of an antibody to bind to its target. For the purposes of the present invention, the term specifically binds' refers to an antibody that binds to a target, such as MCM5, with a binding affinity that is at least 2-fold, 10-fold, 50-fold or 100-fold greater than its binding affinity for binding to another non-target molecule.

Preferably, the first monoclonal antibody and/or the second monoclonal antibody is capable of binding to MCM5 with a binding affinity that is at least 2-fold, 10-fold, 50-fold, or 100-fold greater than its binding affinity for binding to another non-target molecule. Even more preferably, the first monoclonal antibody and/or the second monoclonal antibody is capable of binding to SEQ ID NO: 1 (such as the first monoclonal antibody) or SEQ ID NO: 2 (such as the second monoclonal antibody) with a binding affinity that is at least 2-fold, 10-fold, 50-fold, or 100-fold greater than its binding affinity for binding to another non-target molecule.

A preferred method for the evaluation of binding affinity for MCM5 is by ELISA. Preferably, the first monoclonal antibody and/or the second monoclonal antibody have an affinity for MCM5 (measured as an EC50 or 50% maximum binding concentration, as described in Example 2) of 2500 ng/ml or lower, 1500 ng/ml or lower, 1000 ng/ml or lower, 600 ng/ml or lower, 50 ng/ml or lower, 30 ng/ml or lower, 20 ng/ml or lower, or 10 ng/ml or lower. The EC50 will typically be higher than 1 ng/ml and thus the EC50 may be between 1 ng/ml and any of the upper limits specified in the preceding sentence. Other standard assays to evaluate the binding ability of ligands such as antibodies towards targets are known in the art, including for example, Western blots, RIAs, and flow cytometry analysis. The binding kinetics (e.g. binding affinity) of the antibody also can be assessed by standard assays known in the art, such as by Surface Plasmon Resonance (SPR) (e.g. Biacore™ system) analysis. The affinity constant (KD) for binding to MCM5 is preferably in the range of 0.01-10 nM, 0.01-5 nM, 0.01-1 nM, 0.01-0.5 nM, 0.01-0.25 nM, 0.025-0.25 nM, or 0.04-0.25 nM. In one embodiment, the first and/or second monoclonal antibody used in the ELISA has an affinity for MCM5 of about 0.01 nM, 0.02 nM, 0.03 nM, 0.04 nM, 0.05 nM, 0.06 nM, 0.07 nM, 0.08 nM, 0.09 nM, 0.1 nM, 0.2 nM, 0.3 nM, 0.4 nM, or 0.5 nM. In one embodiment, the first monoclonal antibody and/or second monoclonal antibody used in the ELISA has an affinity for MCM5 of about 0.05 nM. In one embodiment, the first monoclonal antibody and/or second monoclonal antibody used in the ELISA has an affinity for MCM5 of about 0.2 nM. In one embodiment, the first monoclonal antibody and/or second monoclonal antibody used in the ELISA has an affinity for MCM5 of about 0.233 nM. In one embodiment, the first monoclonal antibody has an affinity for MCM5 of about 0.05 nM and the second monoclonal antibody has an affinity for MCM5 of about 0.2 nM. In one embodiment, the first monoclonal antibody has an affinity for MCM5 of about 0.05 nM and the second monoclonal antibody has an affinity for MCM5 of about 0.233 nM. In one embodiment, the first monoclonal antibody is 12A7 and has an affinity for MCM5 of about 0.05 nM and the second monoclonal antibody is 4B4 and has an affinity for MCM5 of about 0.2 nM. In one embodiment, the first monoclonal antibody is 12A7 and has an affinity for MCM5 of about 0.05 nM and the second monoclonal antibody is 4B4 and has an affinity for MCM5 of about 0.233 nM. The association rate (ka) is preferably in the range of 0.4-3.4×10⁶ 1/M. The dissociation rate (kd) is preferably in the range of 1-10×10⁻³ 1/s. These values may typically be determined by SPR (surface plasmon resonance).

Antibody Complementary Determining Regions (CDRs)

The methods and uses of the present invention may comprise the use of a first monoclonal antibody and/or a second monoclonal antibody comprising at least one of the CDRs of antibodies 12A7 or 4B4, i.e. a CDR selected from the group consisting of:
a. 12A7 CDRH1 which has a sequence of SEQ ID NO: 9 or a sequence that differs from SEQ ID NO: 9 by 1, 2 or 3 amino acid substitutions;
b. 12A7 CDRH2 which has a sequence of SEQ ID NO: 11 or a sequence that differs from SEQ ID NO: 11 by 1, 2 or 3 amino acid substitutions;
c. 12A7 CDRH3 which has a sequence of SEQ ID NO: 13 or a sequence that differs from SEQ ID NO: 13 by 1, 2 or 3 amino acid substitutions;
d. 12A7 CDRL1 which has a sequence of SEQ ID NO: 3 or a sequence that differs from SEQ ID NO: 3 by 1, 2 or 3 amino acid substitutions;
e. 12A7 CDRL2 which has a sequence of SEQ ID NO: 5 or a sequence that differs from SEQ ID NO: 5 by 1, 2 or 3 amino acid substitutions;
f. 12A7 CDRL3 which has a sequence of SEQ ID NO: 7 or a sequence that differs from SEQ ID NO: 7 by 1, 2 or 3 amino acid substitutions;
g. 4B4 CDRH1 which has a sequence of SEQ ID NO: 21 or a sequence that differs from SEQ ID NO: 21 by 1, 2 or 3 amino acid substitutions;
h. 4B4 CDRH2 which has a sequence of SEQ ID NO: 23 or a sequence that differs from SEQ ID NO: 23 by 1, 2 or 3 amino acid substitutions;
i. 4B4 CDRH3 which has a sequence of SEQ ID NO: 25 or a sequence that differs from SEQ ID NO: 25 by 1, 2 or 3 amino acid substitutions
j. 4B4 CDRL1 which has a sequence of SEQ ID NO: 15 or a sequence that differs from SEQ ID NO: 15 by 1, 2 or 3 amino acid substitutions;
k. 4B4 CDRL2 which has a sequence of SEQ ID NO: 17 or a sequence that differs from SEQ ID NO: 17 by 1, 2 or 3 amino acid substitutions; and
l. 4B4 CDRL3 which has a sequence of SEQ ID NO: 19 or a sequence that differs from SEQ ID NO: 19 by 1, 2 or 3 amino acid substitutions.

Antibodies that have the same CDRs as the 4B4 and 12A7 antibodies may differ substantially from the sequences of 4B4 and 12A7 in other regions. Such antibodies may, for example, be antibody fragments.

The phrase “sequence that differs from SEQ ID NO: 3 by a single amino acid substitution” refers to the possibility of replacing one amino acid defined in SEQ ID NO: 3 by a different amino acid. Preferably such a replacement is a conservative amino acid substitution. The following eight groups each contain amino acids that are typically conservative substitutions for one another (1) Alanine, Glycine; (2) Aspartic acid, Glutamic acid; (3) Asparagine, Glutamine; (4) Arginine, Lysine; (5) Isoleucine, Leucine, Methionine, Valine; (6) Phenylalanine, Tyrosine, Tryptophan; (7) Serine, Threonine; and (8) Cysteine, Methionine.

In an embodiment, the first monoclonal antibody comprises at least one CDR from the heavy chain of 12A7 (12A7 CDRH1, 12A7 CDRH2 or 12A7 CDRH3) as well as at least one CDR from the light chain of 12A7 (12A7 CDRL1, 12A7 CDRL2 or 12A7 CDRL3). In a further embodiment, the first monoclonal antibody comprises at least two CDRs from the heavy chain of 12A7 and at least two CDRs from the light chain of 12A7. In a preferred embodiment, the first monoclonal antibody comprises all three CDRs from the heavy chain of 12A7 and/or all three CDRs from the light chain of 12A7. In an embodiment, the first monoclonal antibody comprises 12A7 CDRL1 and 12A7 CDRL2, 12A7 CDRL1 and 12A7 CDRL3, 12A7 CDRL1 and 12A7 CDRH1, 12A7 CDRL1 and 12A7 CDRH2, 12A7 CDRL1 and 12A7 CDRH3, 12A7 CDRL2 and 12A7 CDRL3, 12A7 CDRL2 and 12A7 CDRH1, 12A7 CDRL2 and 12A7 CDRH2, 12A7 CDRL2 and 12A7 CDRH3, 12A7 CDRL3 and 12A7 CDRH1, 12A7 CDRL3 and 12A7 CDRH2, 12A7 CDRL3 and 12A7 CDRH3, 12A7 CDRH1 and 12A7 CDRH2, 12A7 CDRH1 and 12A7 CDRH3, or 12A7 CDRH2 and 12A7 CDRH3.

In an embodiment, the second monoclonal antibody comprises at least one CDR from the heavy chain of 4B4 (4B4 CDRH1, 4B4 CDRH2 or 4B4 CDRH3) as well as at least one CDR from the light chain of 4B4 (4B4 CDRL1, 4B4 CDRL2 or 4B4 CDRL3). In a further embodiment, the second monoclonal antibody comprises at least two CDRs from the heavy chain of 4B4 and at least two CDRs from the light chain of 4B4. In a preferred embodiment, the second monoclonal antibody comprises all three CDRs from the heavy chain of 4B4 and/or all three CDRs from the light chain of 4B4. In an embodiment, the second monoclonal antibody comprises 4B4 CDRL1 and 4B4 CDRL2, 4B4 CDRL1 and 4B4 CDRL3, 4B4 CDRL1 and 4B4 CDRH1, 4B4 CDRL1 and 4B4 CDRH2, 4B4 CDRL1 and 4B4 CDRH3, 4B4 CDRL2 and 4B4 CDRL3, 4B4 CDRL2 and 4B4 CDRH1, 4B4 CDRL2 and 4B4 CDRH2, 4B4 CDRL2 and 4B4 CDRH3, 4B4 CDRL3 and 4B4 CDRH1, 4B4 CDRL3 and 4B4 CDRH2, 4B4 CDRL3 and 4B4 CDRH3, 4B4 CDRH1 and 4B4 CDRH2, 4B4 CDRH1 and 4B4 CDRH3, or 4B4 CDRH2 and 4B4 CDRH3. In a preferred embodiment, an antibody comprises at least one CDR having a sequence identical to that described in any one of SEQ ID NO: 3 (12A7 CDRL1), SEQ ID NO: 5 (12A7 CDRL2), SEQ ID NO: 7 (12A7 CDRL3), SEQ ID NO: 9 (12A7 CDRH1), SEQ ID NO: 11 (12A7 CDRH2), SEQ ID NO: 13 (12A7 CDR H3), SEQ ID NO: 15 (4B4 CDRL1), SEQ ID NO: 17 (4B4 CDRL2), SEQ ID NO: 19 (4B4 CDRL3), SEQ ID NO: 21 (4B4 CDRH1), SEQ ID NO: 23 (4B4 CDRH2) or SEQ ID NO: 25 (4B4 CDRH3). In an embodiment, where the first monoclonal antibody comprises 12A7 CDRL2, the 12A7 CDRL2 has the sequence described in SEQ ID NO: 5. In a further embodiment, where the first monoclonal antibody comprises 12A7 CDRL1, the 12A7 CDRL1 has the sequence described in SEQ ID NO: 3. In a further embodiment, where the first monoclonal antibody comprises 12A7 CDRL3, the 12A7 CDRL3 has the sequence described in SEQ ID NO: 7. In a further embodiment, where the first monoclonal antibody comprises 12A7 CDRH1, the 12A7 CDRH1 has the sequence described in SEQ ID NO: 9. In a further embodiment, where the first monoclonal antibody comprises 12A7 CDRH2, the 12A7 CDRH2 has the sequence described in SEQ ID NO: 11. In a further embodiment, where the first monoclonal antibody comprises 12A7 CDRH3, the 12A7 CDRH3 has the sequence described in SEQ ID NO: 13. In a further embodiment, where the second monoclonal antibody comprises 4B4 CDRL1, the 4B4 CDRL1 has the sequence described in SEQ ID NO: 15. In a further embodiment, where the second monoclonal antibody comprises 4B4 CDRL2, the 4B4 CDRL2 has the sequence described in SEQ ID NO: 17. In a further embodiment, where the second monoclonal antibody comprises 4B4 CDRL3, the 4B4 CDRL3 has the sequence described in SEQ ID NO: 19. In a further embodiment, where the second monoclonal antibody comprises 4B4 CDRH1, the 4B4 CDRH1 has the sequence described in SEQ ID NO: 21. In a further embodiment, where the second monoclonal antibody comprises 4B4 CDRH2, the 4B4 CDRH2 has the sequence described in SEQ ID NO: 23. In a further embodiment, where the second monoclonal antibody comprises 4B4 CDRH3, the 4B4 CDRH3 has the sequence described in SEQ ID NO: 25.

Preferably, the first monoclonal antibody and/or the second monoclonal antibody comprises at least one of the CDRs of 12A7 or 4B4 and binds (optionally specifically binds) to MCM5. Even more preferably, the first monoclonal antibody and/or the second monoclonal antibody comprises at least one of the CDRs of 12A7 or 4B4 and binds (optionally specifically binds) to SEQ ID NO: 1 or SEQ ID NO: 2.

Antibody Heavy and Light Chain Variable Regions

In some embodiments, the first monoclonal antibody used in the present invention comprises a heavy chain variable region having a sequence at least 85%, 90%, 95%, 98%, 99% or 100% identical to SEQ ID NO: 29. In some embodiments, the first monoclonal antibody used in the present invention comprises a light chain variable region sequence having a sequence at least 85%, 90%, 95%, 98%, 99% or 100% identical to SEQ ID NO: 27. In some embodiments, the second monoclonal antibody used in the present invention comprises a heavy chain variable region having a sequence at least 85%, 90%, 95%, 98%, 99% or 100% identical to SEQ ID NO: 33. In some embodiments, the second monoclonal antibody used in the present invention comprises a light chain variable region sequence having a sequence at least 85%, 90%, 95%, 98%, 99% or 100% identical to SEQ ID NO: 31. Such antibodies may be referred to as “variant antibodies”.

In an embodiment, the first monoclonal antibody comprises a heavy chain variable region having a sequence at least 95% identical to SEQ ID NO: 29. In a further embodiment, the first monoclonal antibody comprises a heavy chain variable region having a sequence at least 98% identical to SEQ ID NO: 29. In one embodiment, the first monoclonal antibody comprises a light chain variable region having a sequence at least 95% identical to SEQ ID NO: 27. In a further embodiment, the first monoclonal antibody comprises a light chain variable region having a sequence at least 98% identical to SEQ ID NO: 27. In a further embodiment, the first monoclonal antibody comprises a light chain variable region having a sequence at least 95% identical to SEQ ID NO: 27 and a heavy chain variable region having a sequence at least 95% identical to SEQ ID NO: 29. In a preferred embodiment, the first monoclonal antibody comprises a heavy chain variable region having a sequence at least 98% identical to SEQ ID NO: 29 and a light chain variable region having a sequence at least 98% identical to SEQ ID NO: 27.

In a further embodiment, the second monoclonal antibody comprises a heavy chain variable region having a sequence at least 95% identical to SEQ ID NO: 33. In a further embodiment, the second monoclonal antibody comprises a heavy chain variable region having a sequence at least 98% identical to SEQ ID NO: 33. In a further embodiment, the second monoclonal antibody comprises a light chain variable region having a sequence at least 95% identical to SEQ ID NO: 31. In a further embodiment, the second monoclonal antibody comprises a light chain variable region having a sequence at least 98% identical to SEQ ID NO: 31. In a further embodiment, the second monoclonal antibody has a heavy chain variable region having a sequence at least 95% identical to SEQ ID NO:33 and a light chain variable region having a sequence at least 95% identical to SEQ ID NO: 31. In a preferred embodiment, the second monoclonal antibody has a heavy chain variable region having a sequence at least 98% identical to SEQ ID NO: 33 and a light chain variable region having a sequence at least 98% identical to SEQ ID NO: 31.

As is known to the person skilled in the art, antibodies contain multiple regions including framework regions. Deletion or addition of amino acids in the framework regions is unlikely to affect the ability of the antibody to bind to its target. On the other hand, mutations in the CDRs are considerably more likely to affect the ability of an antibody to bind to a target. Thus, in certain embodiments of the invention, variant antibodies have CDRs which are identical to the CDRs of the 12A7 or 4B4 antibodies or have CDRs which vary in only a single amino acid substitution (preferably a conservative amino acid substitution). The first monoclonal antibody and/or the second monoclonal antibody may have framework regions which differ in sequence quite significantly from those described in SEQ ID NO: 27, 29, 31 or 33.

Optionally, where the first monoclonal antibody comprises a heavy chain variable region having a sequence at least 85%, 90%, 95%, 98%, 99% or 100% identical to SEQ ID NO: 29, the antibody further comprises at least one of 12A7 CDRH1, 12A7 CDRH2 or 12A7 CDRH3. It is understood by the person skilled in the art that, since target binding specificity is determined by the CDRs, an antibody comprising the CDRs of 12A7 may still bind to MCM5 even if the remainder of the antibody sequence is quite variable. For this reason where the first monoclonal antibody comprises at least one of 12A7 CDRH1, 12A7 CDRH2 or 12A7 CDRH3 the first monoclonal antibody preferably comprises a heavy chain variable region having a sequence at least 90% identical to SEQ ID NO: 29. In a more preferred embodiment the first monoclonal antibody of the invention comprises 12A7 CDRH1, 12A7 CDRH2 and 12A7 CDRH3 and comprises a heavy chain variable region having a sequence at least 95% identical to SEQ ID NO:29.

Optionally, where the second monoclonal antibody comprises a heavy chain variable region having a sequence at least 85%, 90%, 95%, 98%, 99% or 100% identical to SEQ ID NO: 33, the second monoclonal antibody further comprises at least one of 4B4 CDRH1, 4B4 CDRH2 or 4B4 CDRH3. It is understood by the person skilled in the art that, since target binding specificity is determined by the CDRs, an antibody comprising the CDRs of 4B4 may still bind to MCM5 even if the remainder of the antibody sequence is quite variable. For this reason, where the second monoclonal antibody comprises at least one of 4B4 CDRH1, 4B4 CDRH2 or 4B4 CDRH3 the second monoclonal antibody preferably comprises a heavy chain variable region having a sequence at least 90% identical to SEQ ID NO: 33. In a more preferred embodiment, the second monoclonal antibody comprises 4B4 CDRH1, 4B4 CDRH2 and 4B4 CDRH3 and comprises a heavy chain variable region having a sequence at least 95% identical to SEQ ID NO: 33.

Optionally, where the first monoclonal antibody comprises a light chain variable region having a sequence at least 85%, 90%, 95%, 98%, 99% or 100% identical to SEQ ID NO: 27, the first monoclonal antibody further comprises at least one of 12A7 CDRL1, 12A7 CDRL2 or 12A7 CDRL3. It is understood by the person skilled in the art that, since target binding specificity is determined by the CDRs, an antibody comprising the CDRs of 12A7 may still bind to MCM5 even if the remainder of the antibody sequence is quite variable. For this reason, where the first monoclonal antibody comprises at least one of 12A7 CDRL1, 12A7 CDRL2 or 12A7 CDRL3 the first monoclonal antibody preferably comprises a light chain variable region having a sequence at least 90% identical to SEQ ID NO: 27. In a more preferred embodiment, the first monoclonal antibody comprises 12A7 CDRL1, 12A7 CDRL2 and 12A7 CDRL3 and comprises a light chain variable region having a sequence at least 95% identical to SEQ ID NO: 27.

Optionally, where the second monoclonal antibody of the invention comprises a light chain variable region having a sequence at least 85%, 90%, 95%, 98%, 99% or 100% identical to SEQ ID NO: 31, the second monoclonal antibody further comprises at least one of 4B4 CDRL1, 4B4 CDRL2 or 4B4 CDRL3. It is understood by the person skilled in the art that, since target binding specificity is determined by the CDRs, an antibody comprising the CDRs of 4B4 may still bind to MCM5 even if the remainder of the antibody sequence is quite variable. For this reason, where the second monoclonal antibody comprises at least one of 4B4 CDRL1, 4B4 CDRL2 or 4B4 CDRL3 the second monoclonal antibody preferably comprises a heavy chain variable region having a sequence at least 90% identical to SEQ ID NO: 31. In a more preferred embodiment, the second monoclonal antibody comprises 4B4 CDRL1, 4B4 CDRL2 and 4B4 CDRL3 and comprises a heavy chain variable region having a sequence at least 95% identical to SEQ ID NO:31.

In a further embodiment, the first monoclonal antibody comprises:
(i) 12A7 CDRH1, 12A7 CDRH2 and 12A7 CDRH3;
(ii) a heavy chain variable region having a sequence at least 95% identical to SEQ ID NO: 29;
(iii) 12A7 CDRL1, 12A7 CDRL2 and 12A7 CDRL3; and
(iv) a light chain variable region having a sequence at least 95% identical to SEQ ID NO: 27.

In a further embodiment, the second monoclonal antibody comprises:
(i) 4B4 CDRH1, 4B4 CDRH2 and 4B4 CDRH3;
(ii) a heavy chain variable region having a sequence at least 95% identical to SEQ ID NO: 33;
(iii) 4B4 CDRL1, 4B4 CDRL2 and 4B4 CDRL3; and
(iv) a heavy chain variable region having a sequence at least 95% identical to SEQ ID NO: 31.

An antibody having a heavy chain variable sequence identical to SEQ ID NO: 29 and a light chain variable sequence identical to SEQ ID NO: 27 may be referred to as antibody 12A7. An antibody having a heavy chain variable sequence identical to SEQ ID NO: 33 and a light chain variable sequence identical to SEQ ID NO: 31 may be referred to as an antibody 4B4.

It is well within the knowledge of the person skilled in the art how to make variant antibodies which bind to MCM5. Such variant antibodies may comprise 1, 2, 3, 4, 5, 6, 7, 8, 9, 10, 15, 20, 25, 50 or 100 substitution or deletion mutations compared to SEQ ID NOs: 27, 29, 31 or 33. ‘Deletion’ variant antibodies may comprise the deletion of 1, 2, 3, 4, 5 or more amino acids or, in some cases, the deletion of entire regions of SEQ ID NOs: 27, 29, 31 or 33. ‘Substitution’ variants may comprise the replacement of 1, 2, 3, 4, 5 or more amino acids with the same number of new amino acids.

Preferably, the variant antibodies described herein comprise sequences differing from SEQ ID NOs: 27, 29, 31 or 33 by conservative amino acid substitutions (optionally only by conservative amino acid substitutions). The skilled person is well aware that such conservative substitutions are unlikely to alter the binding properties of an antibody.

In some embodiments, the ELISA assay or sandwich ELISA assay comprises the use of one or more of the monoclonal antibodies described herein. In one embodiment, the ELISA assay or sandwich ELISA assay comprises exposing the urine sample to a first monoclonal antibody described herein. In one embodiment, the ELISA assay or sandwich ELISA assay comprises exposing the urine sample to a second monoclonal antibody described herein. In one embodiment, the ELISA assay or sandwich ELISA assay comprises exposing the urine sample to first monoclonal antibody described herein and/or second monoclonal antibody described herein. In one embodiment, the ELISA assay or sandwich ELISA assay comprises exposing the urine sample to first monoclonal antibody described herein and second monoclonal antibody described herein. In some embodiments, the first monoclonal antibody and/or the second monoclonal antibody used in the ELISA assay or sandwich ELISA assay bind specifically to MCM5.

In some embodiments, the first monoclonal antibody used in the ELISA assay or sandwich ELISA assay binds to SEQ ID NO: 1. In some embodiments, the second monoclonal antibody used in the ELISA assay or sandwich ELISA assay binds to SEQ ID NO: 2. In some embodiments, the first monoclonal antibody used in the ELISA assay or sandwich ELISA assay binds to SEQ ID NO: 1 and the second monoclonal antibody used in the ELISA assay or sandwich ELISA assay binds to SEQ ID NO: 2.

In some embodiment, the first monoclonal antibody used in the ELISA assay or sandwich ELISA comprises at least one of the CDRs of 12A7 described herein. In some embodiments, the second monoclonal antibody used in the ELISA assay or sandwich ELISA assay comprises at least one of the CDRs of 4B4 described herein. In some embodiments, the first monoclonal antibody used in the ELISA assay or sandwich ELISA assay comprises at least one of the CDRs of 12A7 described herein and the second monoclonal antibody used in the ELISA assay or sandwich ELISA assay comprises at least one of the CDRs of 4B4 described herein.

In some embodiments, the first monoclonal antibody used in the ELISA assay or sandwich ELISA is a monoclonal antibody described herein which comprises at least one of the CDRs of 12A7. In some embodiments, the second monoclonal antibody used in the ELISA assay or sandwich ELISA assay is a monoclonal antibody described herein which comprises at least one of the CDRs of 4B4. In some embodiments, the first monoclonal antibody used in the ELISA assay or sandwich ELISA assay is a monoclonal antibody described herein which comprises at least one of the CDRs of 12A7 and the second monoclonal antibody used in the ELISA assay or sandwich ELISA assay is a monoclonal antibody described herein which comprises at least one of the CDRs of 4B4.

In some embodiments, the first monoclonal antibody used in the ELISA assay or sandwich ELISA is a monoclonal antibody described herein which comprises at least one of the CDRs of 12A7 and binds to SEQ ID NO: 1. In some embodiments, the second monoclonal antibody used in the ELISA assay or sandwich ELISA assay is a monoclonal antibody described herein which comprises at least one of the CDRs of 4B4 and binds to SEQ ID NO: 2. In some embodiments, the first monoclonal antibody used in the ELISA assay or sandwich ELISA assay is a monoclonal antibody described herein which comprises at least one of the CDRs of 12A7 and binds to SEQ ID NO: 1 and the second monoclonal antibody used in the ELISA assay or sandwich ELISA assay is a monoclonal antibody which comprises at least one of the CDRs of 4B4 and binds to SEQ ID NO: 2.

In some embodiments, the first monoclonal antibody used in the ELISA assay or sandwich ELISA is 12A7. In some embodiments, the second monoclonal antibody used in the ELISA assay or sandwich ELISA assay is 4B4. In some embodiments, the first monoclonal antibody used in the ELISA assay or sandwich ELISA assay is 12A7 and the second monoclonal antibody used in the ELISA assay or sandwich ELISA assay is 4B4.

Kits

The present invention also provides a kit comprising: (i) a first MCM5 binding agent described herein; (ii) a second MCM5 binding agent described herein; and/or (iii) a lysis buffer capable of releasing MCM5 from cells in a urine sample e.g. a lysis buffer described herein; and (iv) instructions for use of the first MCM5 binding agent, the second MCM5 binding agent, and/or the lysis buffer in: a method of detecting the presence or absence of a recurrent bladder cancer in a subject, a method for diagnosing a subject with a recurrent bladder cancer, and/or a method of monitoring a subject for recurrence of a bladder cancer. The kit can be used to perform the methods of the present invention. The components of the kit may be used to carry out the steps of the methods described herein, for example the lysis buffer can be used to release MCM5 from cells in the urine sample, and the first MCM5 binding agent and/or the second MCM5 binding agent can be used to perform an assay to assess the amount of MCM5 in the sample.

Uses

The present invention provides the use of a first MCM5 binding agent and/or a second MCM5 binding agent for detecting the presence or absence of a recurrent bladder cancer in a subject. The present invention also provides the use of a first MCM5 binding agent and/or a second MCM5 binding agent for monitoring a subject for recurrence of a bladder cancer. The first and second MCM5 binding agents may be used in any suitable method, assay or technique for detecting the presence or absence of a recurrent bladder cancer in a subject or monitoring a subject for recurrence of a bladder cancer. The uses and methods of the present invention may be in vitro or ex-vivo uses or methods.

### Examples

Materials and Methods

A multicentre prospective, double-blinded, cohort study was carried out between August 2017 and July 2019 at 21 centres across Europe. Ethical approval was obtained from all local sites (Research Ethics committee approval reference REC:17.NE.0174). Patients recruited were those:
over 18 years of age;
with a previous diagnosis of urothelial carcinoma, defined as a positive trans urethral resection of bladder tumour (TURBT)/biopsy in the preceding 24 months;
undergoing cystoscopic surveillance;
able to produce 10 mL of urine; and
able to provide informed consent.

Sample Processing

Voided urine samples were collected from these patients and processed. Briefly, a minimum of 10 mL urine was collected from each patient. Urine was agitated to ensure a homogenous mix and between 10 and 50 mL was transferred into a clean centrifuge tube. Samples were centrifuged at room temperature at 1500 g for 5 minutes. Supernatant was discarded to waste taking care not to disturb the cell sediment pellet, tubes were placed upside down on absorbent paper. Cell sediment pellets were resuspended in an appropriate volume of lysis buffer (10 μL per mL of urine) and incubated at room temperature for 1 hr before being stored at less than −20° C. The lysis buffer used comprises 25 mM Tris, 150 mM sodium chloride, 1% sodium deoxycholate, 0.1% SDS and 1% Triton-X100.

MCM5 ELISA

Patient lysates were subjected to an MCM5 ELISA test (ADXBLADDER). Briefly 100 of lysate was added to the MCM5 ELISA micro-titre plate (samples and controls were run in duplicates) and incubated for 60 minutes at room temperature on a plate shaker (700 RPM), following incubation wells were washed 6 times with 350 μL of 1× wash buffer using an automated plate washer. 100 μL of MCM5-HRP conjugated antibody was added to each well and incubated at room temperature for 30 minutes prior to being washed 6 times with 350 μl of 1× wash buffer as above. 100 μL of TMB was added to each well and incubated for 30 minutes in the dark prior to the addition of a stop solution (0.5 M H₂SO₄). Optical density (OD) was measured at 450 nm and 630 nm (reference wavelength). Amounts of MCM5 were calculated using a positive control of known recombinant MCM5 control (1.5 mg/ml) and a negative control (Lysis Buffer).

The results of the MCM5 ELISA were then compared to the diagnosis obtained by cystoscopy and pathological results of TURBT/biopsy of suspect lesions.

Results

Out of 1192 eligible patients enrolled, 120 were diagnosed with bladder cancer (10.1% prevalence; LGpTa). Of these 120 cancers, 95 were Low Grade pTa (a non-invasive papillary carcinoma) tumours. Overall sensitivity of the MCM5 ELISA for recurrent bladder cancer was 45% (95% CI: 35.9% to 54.4%). Sensitivity for all non-low grade (LG) pTa tumours was very high at 84% (95% CI: 63.9% to 95.5%). Specificity was 72% (95% CI 69.2% to 74.7%). Overall negative predictive value (NPV) of the MCM5 ELISA for recurrent bladder cancer was 92% (95% CI: 90.8% to 93.3%). Moreover, the MCM5 ELISA was able to rule out all non-LGpTa recurrent tumours with a NPV of 99.5% (95% CI: 98.7% to 99.8%). These data are summarized in Table 1 below.

TABLE 1
Statistical performance of MCM5 ELISA

| Parameter | n/N | Result (%) | 95% CI (%) |
|---|---|---|---|
| Overall sensitivity^{a} | 54/120^{a} | 45 | 35.9 to 54.4 |
| Overall specificity^{b} | 772/1072 | 72 | 69.2 to 74.7 |
| Overall NPV^{c} | 772/838 | 92 | 90.8 to 93.3 |
| Overall PPV^{d} | 54/354 | 15.3 | 12.6 to 18.3 |
| Sensitivity^{a} excluding low grade | 21/25 | 84.0 | 63.9 to 95.5 |
| non-invasive papillary | | | |
| carcinoma (LGpTa) | | | |
| NPV^{c} excluding low | 834/838 | 99.5 | 98.7 to 99.8 |
| grade non-invasive | | | |
| papillary carcinoma (LGpTa) | | | |

^{a}proportion of subjects having a recurrent bladder cancer correctly identified as such
^{b}proportion of subject not having a recurrent bladder cancer correctly identified as such
^{c}proportion of subjects not having a recurrent cancer divided by the total number of negative test results
^{d}proportion of subjects having a recurrent bladder cancer divided by the total number of positive test results

It was then confirmed that the MCM5 ELISA has a particularly high sensitivity—greater than 80%—for high grade (HG) recurrent bladder cancer. These data are summarized in Table 2 below. It was also shown that the MCM5 ELISA has high sensitivity— 75% or greater—towards carcinoma in situ (CIS), high grade noninvasive papillary carcinoma (HGpTa) and T1 bladder cancer.

TABLE 2
Sensitivity of MCM5 ELISA by bladder cancer subtype

| | | Sensitivity | |
|---|---|---|---|
| Cancer sub-type | n/N | (%) | 95% CI (%) |
| Non-invasive papillary carcinoma (pTa) | 26/70 | 37.1 | 25.9 to 49.5 |
| Low grade pTa (LGpTa) | 16/57 | 28.1 | 17.0 to 41.5 |
| High grade pTa (HGpTa) | 10/12 | 83.3 | 51.6 to 97.9 |
| Cancer has invaded the | 5/6 | 83.3 | 35.9 to 99.6 |
| connective tissue layer | | | |
| (lamina propria) of the | | | |
| bladder wall (pTI) | | | |
| Carcinoma in situ (CIS) | 6/8 | 75.0 | 34.9 to 96.8 |
| Low Grade (LG) | 16/57 | 28.1 | 17.0 to 41.5 |
| High Grade (HG) | 19/23 | 82.6 | 61.22 to 95.1 |

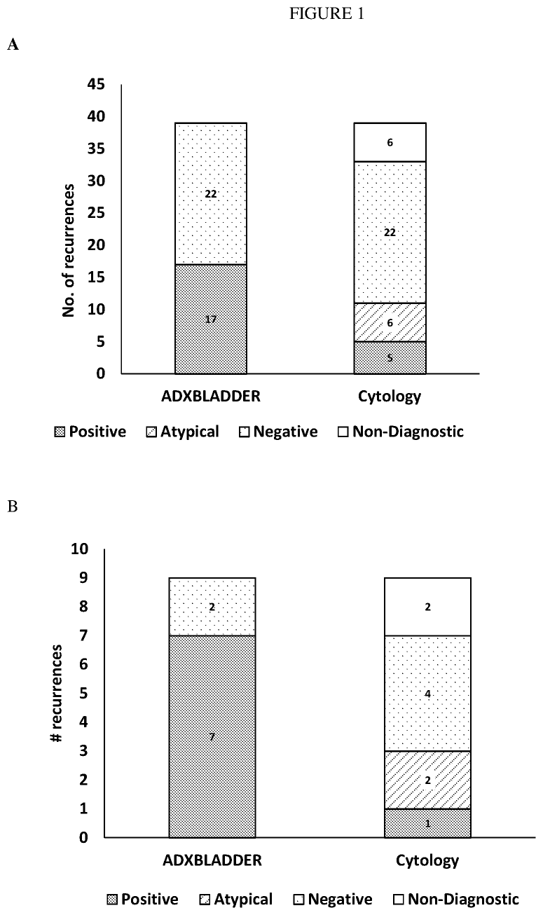

Matching cytology was performed on 362 of the patients, of which 39 were diagnosed with bladder cancer. In this population the sensitivity of cytology was just 17%. Of all the tumours with matching cytology data the MCM5 ELISA detected 17/39 of the tumours while cytology detected just 5/39 tumours. Furthermore, the MCM5 ELISA detected 7/9 non-LGpTa tumours whilst cytology only detected 1/9 non-LGpTa tumours. These data are summarized in FIGS. 1A and 1B. All tumours detected by cytology were detected by the MCM5 ELISA.

All publications mentioned in the above specification are herein incorporated by reference. Various modifications and variations of the described aspects and embodiments of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the following claims.

TABLE 3
Sequences

| SEQ ID NO | Nucleotide/Polypeptide |
|---|---|
| 1 | WDETKGE (epitope to which antibody 12A7 binds) |
| 2 | DDRVAIH (epitope to which antibody 4B4 binds |
| 3 | 12A7 light chain CDR 1 polypeptide sequence |
| 4 | 12A7 light chain CDR 1 nucleotide sequence |
| 5 | 12A7 light chain CDR 2 polypeptide sequence |
| 6 | 12A7 light chain CDR 2 nucleotide sequence |
| 7 | 12A7 light chain CDR 3 polypeptide sequence |
| 8 | 12A7 light chain CDR 3 nucleotide sequence |
| 9 | 12A7 heavy chain CDR 1 polypeptide sequence |
| 10 | 12A7 heavy chain CDR 1 nucleotide sequence |
| 11 | 12A7 heavy chain CDR 2 polypeptide sequence |
| 12 | 12A7 heavy chain CDR 2 nucleotide sequence |
| 13 | 12A7 heavy chain CDR 3 polypeptide sequence |
| 14 | 12A7 heavy chain CDR 3 nucleotide sequence |
| 15 | 4B4 light chain CDR 1 polypeptide sequence |
| 16 | 4B4 light chain CDR 1 nucleotide sequence |
| 17 | 4B4 light chain CDR 2 polypeptide sequence |
| 18 | 4B4 light chain CDR 2 nucleotide sequence |
| 19 | 4B4 light chain CDR 3 polypeptide sequence |
| 20 | 4B4 light chain CDR 3 nucleotide sequence |
| 21 | 4B4 heavy chain CDR 1 polypeptide sequence |
| 22 | 4B4 heavy chain CDR 1 nucleotide sequence |
| 23 | 4B4 heavy chain CDR 2 polypeptide sequence |
| 24 | 4B4 heavy chain CDR 2 nucleotide sequence |
| 25 | 4B4 heavy chain CDR 3 polypeptide sequence |
| 26 | 4B4 heavy chain CDR 3 nucleotide sequence |
| 27 | 12A7 full light chain variable region sequence |
| | (polypeptide) |
| 28 | 12A7 full light chain variable region sequence |
| | (nucleotide) |
| 29 | 12A7 full heavy chain variable region sequence |
| | (polypeptide) |
| 30 | 12A7 full heavy chain variable region sequence |
| | (nucleotide) |
| 31 | 4B4 full light chain variable region sequence |
| | (polypeptide) |
| 32 | 4B4 full light chain variable region sequence |
| | (nucleotide) variable region |
| 33 | 4B4 full heavy chain variable region sequence |
| | (polypeptide) |
| 34 | 4B4 full heavy chain variable region sequence |
| | (nucleotide) |
| 35 | MCM5 polypeptide sequence |
| 36 | MCM5 polynucleotide sequence |

| Sequence Listing | |
|---|---|
| SEQ ID NO: 1 | |
| WDETKGE | |
| SEQ ID NO: 2 | |
| DDRVAIH | |
| SEQ ID NO: 3 | |
| QNLVQSNGNTY | |
| SEQ ID NO: 4 | |
| CAGAACCTTGTACAAAGTAATGGAAACACCTATTTA | |
| SEQ ID NO: 5 | |
| KVS | |
| SEQ ID NO: 6 | |
| AAGTTTCCAA | |
| SEQ ID NO: 7 | |
| SQSTRVPYT | |
| SEQ ID NO: 8 | |
| TCTCAAAGTACACGTGTTCCGTACACA | |
| SEQ ID NO: 9 | |
| GFSLSTSGMG | |
| SEQ ID NO: 10 | |
| GGGTTTTCACTGAGCACTTCTGGTATGGGT | |
| SEQ ID NO: 11 | |
| IFWDDDK | |
| SEQ ID NO: 12 | |
| ATTTTCTGGGATGATGACAAG | |
| SEQ ID NO: 13 | |
| ARRSDYNYYSMDY | |
| SEQ ID NO: 14 | |
| GCGCGGCGAAGTGACTACAATTACTACTCTATGGACTAC | |
| SEQ ID NO: 15 | |
| QDIGSS | |
| SEQ ID NO: 16 | |
| CAGGACATTGGTAGTAGC | |
| SEQ ID NO: 17 | |
| ATS | |
| SEQ ID NO: 18 | |
| GCCACATCC | |
| SEQ ID NO: 19 | |
| LQYASSPPT | |
| SEQ ID NO: 20 | |
| CTACAATATGCTAGTTCTCCTCCGACG | |
| SEQ ID NO: 21 | |
| GFTFSNYA | |
| SEQ ID NO: 22 | |
| GGATTCACTTTCAGTAACTATGCC | |
| SEQ ID NO: 23 | |
| ISRGGSYT | |
| SEQ ID NO: 24 | |
| ATTAGTCGTGGTGGTAGTTACACC | |
| SEQ ID NO: 25 | |
| ARHGYNYDDGAWFAN | |
| SEQ ID NO: 26 | |
| GCAAGACATGGATATAATTACGACGACGGGGCCTGGTTTGCTAAC | |
| SEQ ID NO: 27 | |
| DIMLTQSPLSLSVTLGDQASISCRSSQNLVQSNGNTYLTWYLQKPGQSPKVLINKVSNRFYGVP | |
| DRFSGSGSGTDFTLRISRVEAEDLGIYFCSQSTRVPYTFGGGTKLEIRR | |
| SEQ ID NO: 28 | |
| GATATCATGCTGACCCAATCTCCACTCTCCCTGTCTGTCACTCTTGGAGATCAGGCCTCCATCT | |
| CTTGCAGATCTAGTCAGAACCTTGTACAAAGTAATGGAAACACCTATTTAACTTGGTACCTGCA | |
| GAAGCCAGGCCAGTCTCCAAAGGTCCTGATCAACAAAGTTTCCAACCGATTTTATGGGGTCCCA | |
| GACAGGTTCAGTGGCAGTGGATCAGGGACAGATTTCACACTCAGGATCAGCAGAGTGGAGGCTG | |
| AGGATCTGGGAATTTATTTCTGCTCTCAAAGTACACGTGTTCCGTACACATTCGGAGGGGGGAC | |
| CAAGCTGGAAATAAGACG | |
| SEQ ID NO: 29 | |
| QQDLQQSGPGILQPTQTLSLTCSFSGFSLSTSGMGVSWIRQSSNMGLEWLAHIFWDDDKRYNPS | |
| LRSRLTLSKDTSSSQVFLMITSVSTADSATYYCARRSDYNYYSMDYWGQGTAVTVSS | |
| SEQ ID NO: 30 | |
| CAGCAAGATCTGCAGCAGTCTGGCCCTGGGATATTGCAGCCCACCCAGACCCTCAGTCTGACTT | |
| GTTCTTTCTCTGGGTTTTCACTGAGCACTTCTGGTATGGGTGTGAGTTGGATTCGTCAATCTTC | |
| AAATATGGGTCTGGAGTGGCTGGCACACATTTTCTGGGATGATGACAAGCGCTATAATCCCTCC | |
| CTGAGGAGCCGACTCACGCTCTCCAAGGATACCTCCAGTAGCCAGGTATTTCTCATGATCACCA | |
| GTGTGAGTACTGCAGATTCTGCCACATACTACTGTGCGCGGCGAAGTGACTACAATTACTACTC | |
| TATGGACTACTGGGGTCAAGGAACCGCAGTCACCGTCTCCTCA | |
| SEQ ID NO: 31 | |
| DIMLTQSPSSLSASLGERVSLTCRASQDIGSSLNWLQQEPDGTIKRLIYATSSLDSGVPKRFSG | |
| SRSGSDYSLTISSLESEDFVDYYCLQYASSPPTFGGGTKLEIK | |
| SEQ ID NO: 32 | |
| GATATCATGCTGACCCAATCTCCATCCTCCTTATCTGCCTCTCTGGGAGAAAGAGTCAGTCTCA | |
| CTTGTCGGGCAAGTCAGGACATTGGTAGTAGCTTAAACTGGCTTCAACAGGAACCAGATGGAAC | |
| TATTAAACGCCTAATCTACGCCACATCCAGTTTAGATTCTGGTGTCCCCAAAAGGTTCAGTGGC | |
| AGTAGGTCTGGGTCAGATTATTCTCTCACCATCAGCAGCCTTGAGTCTGAAGATTTTGTAGACT | |
| ATTACTGTCTACAATATGCTAGTTCTCCTCCGACGTTCGGTGGAGGCACCAAGCTGGAAATCAA | |
| AC | |
| SEQ ID NO: 33 | |
| VKLQESGGGLVKPGGSLKLSCAASGFTFSNYAMSWVRQNPEKRLEWVATISRGGSYTYYPDSVK | |
| GRFTISRDNAKNTLYLQMNSLRSEDTAMYFCARHGYNYDDGAWFANWGQGTLVTVSA | |
| SEQ ID NO: 34 | |
| TAGGTGAAACTGCAGGAGTCTGGGGGAGGCTTAGTGAAGCCTGGAGGGTCCCTGAAACTCTCCT | |
| GTGCAGCCTCTGGATTCACTTTCAGTAACTATGCCATGTCTTGGGTTCGCCAGAATCCGGAGAA | |
| GAGGCTGGAGTGGGTCGCAACCATTAGTCGTGGTGGTAGTTACACCTACTATCCAGACAGTGTG | |
| AAGGGTCGATTCACCATCTCCAGAGACAATGCCAAGAACACCCTGTATCTGCAAATGAACAGTC | |
| TGAGGTCTGAGGACACGGCCATGTATTTCTGTGCAAGACATGGATATAATTACGACGACGGGGC | |
| CTGGTTTGCTAACTGGGGCCAAGGGACTCTGGTCACTGTCTCTGCA | |
| SEQ ID NO: 35 | |
| 10 20 30 40 50 60 | |
| MSGFDDPGIF YSDSFGGDAQ ADEGQARKSQ LQRRFKEFLR QYRVGTDRTG FTFKYRDELK | |
| 70 80 90 100 110 120 | |
| RHYNLGEYWI EVEMEDLASF DEDLADYLYK QPAEHLQLLE EAAKEVADEV TRPRPSGEEV | |
| 130 140 150 160 170 180 | |
| LQDIQVMLKS DASPSSIRSL KSDMMSHLVK IPGIIIAASA VRAKATRISI QCRSCRNTLT | |
| 190 200 210 220 230 240 | |
| NIAMRPGLEG YALPRKCNTD QAGRPKCPLD PYFIMPDKCK CVDFQTLKLQ ELPDAVPHGE | |
| 250 260 270 280 290 300 | |
| MPRHMQLYCD RYLCDKVVPG NRVTIMGIYS IKKFGLTTSR GRDRVGVGIR SSYIRVLGIQ | |
| 310 320 330 340 350 360 | |
| VDTDGSGRSF AGAVSPQEEE EFRRLAALPN VYEVISKSIA PSIFGGTDMK KAIACLLFGG | |
| 370 380 390 400 410 420 | |
| SRKRLPDGLT RRGDINLLML GDPGTAKSQL LKFVEKCSPI GVYTSGKGSS AAGLTASVMR | |
| 430 440 450 460 470 480 | |
| DPSSRNFIME GGAMVLADGG VVCIDEFDKM REDDRVAIHE AMEQQTISIA KAGITTTLNS | |
| 490 500 510 520 530 540 | |
| RCSVLAAANS VFGRWDETKG EDNIDFMPTI LSRFDMIFIV KDEHNEERDV MLAKHVITLH | |
| 550 560 570 580 590 600 | |
| VSALTQTQAV EGEIDLAKLK KFIAYCRVKC GPRLSAEAAE KLKNRYIIMR SGARQHERDS | |
| 610 620 630 640 650 660 | |
| DRRSSIPITV RQLEAIVRIA EALSKMKLQP FATEADVEEA LRLFQVSTLD AALSGTLSGV | |
| 670 680 690 700 710 720 | |
| EGFTSQEDQE MLSRIEKQLK RRFAIGSQVS EHSIIKDFTK QKYPEHAIHK VLQLMLRRGE | |
| 730 | |
| IQHRMQRKVL YRLK | |
| Homo sapiens minichromosome maintenance complex component 5 (MCM5), | |
| mRNA NCBI Reference Sequence: NM 0067393 | |
| SEQ ID NO: 36 | |
| 1 | ggaaaaccag aggcgcagtc atgtcgggat tcgacgatcc tggcattttc tacagcgaca |
| 61 | gcttcggggg cgacgcccag gccgacgagg ggcaggcccg caaatcgcag ctgcagaggc |
| 121 | gcttcaagga gttcctgcgg cggtaccgag tgggcaccga ccgcacgggc ttcaccttca |
| 181 | aatacaggga tgaactcaag cggcattaca acctggggga gtactggatt gaggtggaga |
| 241 | tggaggatct ggccagcttt gatgaggacc tggccgacta cttgtacaag cagccagccg |
| 301 | agcacctgca gctgctggag gaagctgcca aggaggtagc tgatgaggtg acccggcccc |
| 361 | ggccttctgg ggaggaggtg ctccaggaca tccaggtcat gctcaagtcg gacgccagcc |
| 421 | cttccagcat tcgtagcctg aagtcggaca tgatgtcaca cctggtgaag atccctggca |
| 481 | tcatcatcgc ggcctctgcg gtccgtgcca aggccacccg catctctatc cagtgccgca |
| 541 | gctgccgcaa caccctcacc aacattgcca tgcgccctgg cctcgagggc tatgccctgc |
| 601 | ccaggaagtg caacacagat caggctgggc gccccaaatg cccattggac ccgtacttca |
| 661 | tcatgcccga caaatgcaaa tgcgtggact tccagaccct gaagctgcag gagctgcctg |
| 721 | atgcagtccc ccacggggag atgcccagac acatgcagct ctactgcgac aggtacctgt |
| 781 | gtgacaaggt cgtccctggg aacagggtta ccatcatggg catctactcc atcaagaagt |
| 841 | ttggcctgac taccagcagg ggccgtgaca gggtgggcgt gggcatccga agctcctaca |
| 901 | tccgtgtcct gggcatccag gtggacacag atggctctgg ccgcagcttt gctggggccg |
| 961 | tgagccccca ggaggaggag gagttccgtc gcctggctgc cctcccaaat gtctatgagg |
| 1021 | tcatctccaa gagcatcgcc ccctccatct ttgggggcac agacatgaag aaggccattg |
| 1081 | cctgcctgct ctttgggggc tcccgaaaga ggctccctga tggacttact cgccgaggag |
| 1141 | acatcaacct gctgatgcta ggggaccctg ggacagccaa gtcccagctt ctgaagtttg |
| 1201 | tggagaagtg ttctcccatt ggggtataca cgtctgggaa aggcagcagc gcagctggac |
| 1261 | tgacagcctc ggtgatgagg gacccttcgt cccggaattt catcatggag ggcggagcca |
| 1321 | tggtcctggc cgatggtggg gtcgtctgta ttgacgagtt tgacaagatg cgagaagatg |
| 1381 | accgtgtggc aatccacgaa gccatggagc agcagaccat ctctatcgcc aaggctggga |
| 1441 | tcaccaccac cctgaactcc cgctgctccg tcctggctgc tgccaactca gtgttcggcc |
| 1501 | gctgggatga gacgaagggg gaggacaaca ttgacttcat gcccaccatc ttgtcgcgct |
| 1561 | tcgacatgat cttcatcgtc aaggatgagc acaatgagga gagggatgtg atgctggcca |
| 1621 | agcatgtcat cactctgcac gtgagcgcac tgacacagac acaggctgtg gagggcgaga |
| 1681 | ttgacctggc caagctgaag aagtttattg cctactgccg agtgaagtgt ggcccccggc |
| 1741 | tgtcagcaga ggctgcagag aaactgaaga accgctacat catcatgcgg agcggggccc |
| 1801 | gtcagcacga gagggacagt gaccgccgct ccagcatccc catcactgtg cggcagctgg |
| 1861 | aggccattgt gcgcatcgcg gaagccctca gcaagatgaa gctgcagccc ttcgccacag |
| 1921 | aggcagatgt ggaggaggcc ctgcggctct tccaagtgtc cacgttggat gctgccttgt |
| 1981 | ccggtaccct gtcaggggtg gagggcttca ccagccagga ggaccaggag atgctgagcc |
| 2041 | gcatcgagaa gcagctcaag cgccgctttg ccattggctc ccaggtgtct gagcacagca |
| 2101 | tcatcaagga cttcaccaag cagaaatacc cggagcacgc catccacaag gtgctgcagc |
| 2161 | tcatgctgcg gcgcggcgag atccagcatc gcatgcagcg caaggttctc taccgcctca |
| 2221 | agtgagtcgc gccgcctcac tggactcatg gactcgccca cgcctcgccc ctcctgccgc |
| 2281 | tgcctgccat tgacaatgtt gctgggacct ctgcctcccc actgcagccc tcgaacttcc |
| 2341 | caggcaccct cctttctgcc ccagaggaag gagctgtagt gtcctgctgc ctctgggcgc |
| 2401 | ccgcctctag cgcggttctg ggaagtgtgc ttttggcatc cgttaataat aaagccacgg |
| 2461 | tgtgttcagg taaaaaaaaa aaaaaaaaaa aaaaaaaaaa aaaaaaaaaa aaaaaaaaaa |
| 2521 | aaaaaaaaaa aaaa |

1. A method for detecting the presence or absence of a recurrent bladder cancer in a subject, the method comprising:
providing a urine sample previously isolated from the subject;
performing an assay to assess the amount of MCM5 in the sample;
comparing the amount of MCM5 in the sample to a reference;
determining the presence or absence of the recurrent bladder cancer in the subject based on the amount of MCM5 in the sample;
wherein the method does not comprise cytological analysis of the subject or a sample obtained from the subject.

2. The method of claim 1, wherein the recurrent bladder cancer is determined to be present if the amount of MCM5 in the sample is higher than the reference or the recurrent bladder cancer is determined to be absent if the amount of MCM5 in the sample is lower than the reference.

3. A method for diagnosing a subject with a recurrent bladder cancer, the method comprising:
providing a urine sample previously isolated from the subject;
performing an assay to assess the amount of MCM5 in the sample;
comparing the amount of MCM5 in the sample to a reference;
diagnosing the subject with a recurrent bladder cancer if the amount of MCM5 measured in the sample is higher than the reference or diagnosing the subject as not having a recurrent bladder cancer if the amount of MCM5 measured in the sample is lower than the reference;
wherein the method does not comprise cytological analysis of the subject or a sample obtained from the subject.

4. A method for monitoring a subject for recurrence of a bladder cancer, the method comprising:
(a) providing a urine sample previously isolated from the subject;
(b) performing an assay to assess the amount of MCM5 in the sample;
(c) comparing the amount of MCM5 in the sample to a reference;
(d) determining whether the subject does or does not have a recurrent bladder cancer based on the amount of MCM5 in the sample;
(e) repeating steps (a)-(d), optionally wherein steps (a)-(d) are repeated every month or are repeated once every 2, 3, 4, 5, 6, 7, 8, 9, 10, 11, 12, 18, 24, or 36 months;
wherein the method does not comprise cytological analysis of the subject or a sample obtained from the subject.

5. The method of any one of the preceding claims, wherein the step of performing an assay to assess the amount of MCM5 in the sample comprises:
exposing the sample to a lysis buffer capable of releasing MCM5 from cells in the sample in order to release MCM5 from cells in the sample, optionally wherein exposing the sample to the lysis buffer comprises:
passing the sample through a filter for capturing cells, such that cells are captured in the filter; and
(i) passing the lysis buffer through the filter, such that the captured cells are exposed to the lysis buffer; and/or
(ii) incubating the filter in contact with the lysis buffer for a period of time, such that the lysis buffer causes the captured cells to release the MCM5.

6. The method of claim 5, wherein the lysis buffer does not denature an antibody.

7. The method of claim 5 or 6, wherein:
(a) the lysis buffer comprises a detergent;
(b) the lysis buffer comprises a detergent which comprises Triton X-100;
(c) the lysis buffer comprises a detergent which comprises Triton X-100 at a concentration between 0.01% and 25%, between 0.01% and 10%, between 0.05% and 5%, between 0.1% and 2%, between 0.5% and 2%, between 0.75% and 1.25%, or about 1%;
(d) the lysis buffer comprises a detergent which comprises or consists of sodium deoxycholate;
(e) the lysis buffer comprises a detergent which comprises or consists of sodium deoxycholate at a concentration between 0.1% and 20%, between 0.1 and 10%, between 0.1 and 5%, between 0.5% and 5%, between 0.5% and 2.5%, between 0.75% and 2.5%, between 0.75% and 1.25%, or about 1%;
(f) the lysis buffer comprises a detergent which comprises or consists of sodium dodecyl sulphate (SDS);
(g) the lysis buffer comprises a detergent which comprises or consists of sodium dodecyl sulphate (SDS) at a concentration between 0.001% and 10%, between 0.01% and 5%, between 0.05% and 5%, between 0.01% and 1%, between 0.05% and 1%, between 0.05% and 0.5%, between 0.075% and 0.25%, or about 0.1%;
(h) the lysis buffer comprises a detergent which consists of Triton X-100, sodium deoxycholate, and sodium dodecyl sulphate (SDS);
(i) the lysis buffer comprises a detergent which consists of between 0.5% and 2% of Triton X-100, between 0.5% and 2% of sodium deoxycholate, and between 0.05% and 0.5% of sodium dodecyl sulphate (SDS);
(j) the lysis buffer comprises a buffer component;
(k) the lysis buffer comprises a buffer component which has a pH of between pH 4 and pH 9, between pH 5 and pH 8.5, between pH 6 and pH 8, between pH 6.5 and pH 8, between pH 7 and pH 8, between pH 7.3 and pH 7.9, between pH 7.4 and pH 7.8, between pH 7.5 and pH 7.7, or about pH 7.6; and/or maintains the pH of the lysis buffer at between pH 4 and pH 9, between pH 5 and pH 8.5, between pH 6 and pH 8, between pH 6.5 and pH 8, between pH 7 and pH 8, between pH 7.3 and pH 7.9, between pH 7.4 and pH 7.8, between pH 7.5 and pH 7.7, or about pH 7.6;
(l) the lysis buffer comprises a buffer component which comprises or consists of Tris;
(m) the lysis buffer comprises a buffer component which comprises or consists of Tris at a concentration greater than 1 mM, between 1 mM and 350 mM, between 5 and 200 mM, between 5 and 100 mM, between 5 and 50 mM, between 5 mM and 40 mM, between 5 mM and 35 mM, between 10 mM and 35 mM, between 15 mM and 35 mM, between 15 mM and 30 mM, between 20 mM and 30 mM, or about 25 mM;
(n) the lysis buffer comprises a buffer component which consists of Tris at a concentration of between 15 mM and 35 mM;
(o) the lysis buffer comprises a salt;
(p) the lysis buffer comprises sodium chloride;
(q) the lysis buffer comprises sodium chloride at a concentration between 10 mM and 350 mM, between 20 mM and 300 mM, between 50 mM and 250 mM, between 100 mM and 250, between 100 mM and 200 mM, between 125 mM and 175 mM, or about 150 mM;
(r) the lysis buffer comprises sodium chloride at a concentration of between 100 mM and 200 mM;
(s) the lysis buffer comprises:
(i) between 1 mM and 100 mM Tris;
(ii) between 50 mM and 300 mM sodium chloride;
(iii) between 0.1 and 5% sodium deoxycholate;
(iv) between 0.01 and 1% sodium dodecyl sulphate; and/or
(v) between 0.1 and 5% Triton-X100;
(t) the lysis buffer comprises:
(i) between 10 mM and 40 mM Tris;
(ii) between 100 mM and 200 mM sodium chloride;
(iii) between 0.5 and 2% sodium deoxycholate;
(iv) between 0.05 and 0.5% sodium dodecyl sulphate; and/or
(v) between 0.5 and 2% Triton-X100; and/or
(u) the lysis buffer comprises:
(i) about 25 mM Tris;
(ii) about 150 mM sodium chloride;
(iii) about 1% sodium deoxycholate;
(iv) about 0.1% sodium dodecyl sulphate; and/or
(v) about 1% Triton-X100.

8. Use of a first MCM5 binding agent and/or a second MCM5 binding agent for detecting the presence or absence of a recurrent bladder cancer in a subject.

9. Use of a first MCM5 binding agent and/or a second MCM5 binding agent for monitoring a subject for recurrence of a bladder cancer.

10. A first MCM5 binding agent and/or a second MCM5 binding agent for use in a method of diagnosing a subject with a recurrent bladder cancer, optionally wherein the method is as defined in claim 3.

11. The method of any one of claims 1-7, or the use of claim 8 or 9, or the first MCM5 binding agent and/or the second MCM5 binding agent for use according to claim 10, wherein the method or use has:
(i) a higher sensitivity or negative predictive value (NPV) for the recurrent bladder cancer than would be achieved by cytological analysis of the subject or a sample obtained from the subject; and/or
(ii) a sensitivity of at least about 40% and/or an NPV of at least about 85%.

12. The method of any one of claims 1-7, or the use of claim 8 or 9, or the first MCM5 binding agent and/or the second MCM5 binding agent for use according to claim 10, wherein the recurrent bladder cancer is not a low grade non-invasive papillary carcinoma (LGpTa).

13. The method or use of claim 12, or the first MCM5 binding agent and/or the second MCM5 binding agent for use according to claim 12, wherein the method or use has a sensitivity of at least about 80% and/or an NPV of at least about 95%.

14. The method of any one of claim 1-7 or 11-13, the use of any one of claim 8-9 or 11-13, or the first MCM5 binding agent and/or the second MCM5 binding agent for use according any one of claims 10-13, wherein the recurrent bladder cancer is:
a high grade bladder cancer, optionally wherein the method or use has a sensitivity of at least about 75%;
(ii) a high grade non-invasive papillary carcinoma (HGpTa), optionally wherein the method or use has a sensitivity of at least about 75%;
(iii) a carcinoma in situ (CIS; Tis), optionally wherein the method or use has a sensitivity of at least about 70%; or
(iv) is a T1 bladder cancer, optionally wherein the method or use has a sensitivity of at least about 75%.

15. The method of any one of claims 1-7 and 11-14, wherein the assay to assess the amount of MCM5 in the sample is an enzyme-linked immunoabsorbent assay (ELISA), optionally wherein the ELISA is a sandwich ELISA.

16. The use of any one of claim 8-9 or 11-14, or the first MCM5 binding agent and/or the second MCM5 binding agent for use according to any one of claims 10-14, which comprises performing an assay to assess the amount of in a urine sample previously obtained from the subject, optionally wherein assay is an enzyme-linked immunoabsorbent assay (ELISA), optionally wherein the ELISA is a sandwich ELISA.

17. The method of any one of claims 1-7 and 11-15, or the use of claim 16, or the first MCM5 binding agent and/or the second MCM5 binding agent for use according to any one of claim 10-14 or 16, wherein the step of performing an assay to assess the amount of MCM5 in the sample comprises:
exposing the sample to a first MCM5 binding agent and/or a second MCM5 binding agent.

18. The method or use of claim 17, or the first MCM5 binding agent and/or the second MCM5 binding agent for use according to any one of claim 10-14 or 16-17, wherein the first MCM5 binding agent is a first monoclonal antibody which binds to MCM5 and/or the second MCM5 binding agent is a second monoclonal antibody which binds to MCM5, optionally wherein the first and second monoclonal antibodies bind to different epitopes of MCM5.

19. The method or use of claim 18, or the first MCM5 binding agent and/or the second MCM5 binding agent for use according to claim 18, wherein the first monoclonal antibody:
(i) binds to a polypeptide having an amino acid sequence of SEQ ID NO: 1; and/or
(ii) comprises at least one Complementary Determining Region (CDR) selected from the group consisting of:
(a) 12A7 CDRH1 which has a sequence of SEQ ID NO: 9 or a sequence that differs from SEQ ID NO:9 by a single amino acid substitution;
(b) 12A7 CDRH2 which has a sequence of SEQ ID NO: 11 or a sequence that differs from SEQ ID NO:11 by a single amino acid substitution;
(c) 12A7 CDRH3 which has a sequence of SEQ ID NO: 13 or a sequence that differs from SEQ ID NO: 13 by a single amino acid substitution;
(d) 12A7 CDRL1 which has a sequence of SEQ ID NO: 3 or a sequence that differs from SEQ ID NO: 3 by a single amino acid substitution;
(e) 12A7 CDRL2 which has a sequence of SEQ ID NO: 5 or a sequence that differs from SEQ ID NO: 5 by a single amino acid substitution; and
(f) 12A7 CDRL3 which has a sequence of SEQ ID NO: 7 or a sequence that differs from SEQ ID NO: 7 by a single amino acid substitution; and/or
(iii) comprises a heavy chain variable region having a sequence at least 85%, 90%, 95%, 98%, 99%, or 100% identical to SEQ ID NO: 29; and/or
(iv) comprises a light chain variable region sequence having a sequence at least 85%, 90%, 95%, 98%, 99%, or 100% identical to SEQ ID NO: 27; or
(v) competes with the antibody of (i), (ii), (iii), or (iv), and/or the second monoclonal antibody:
(i) binds to a polypeptide having an amino acid sequence of SEQ ID NO: 2; and/or
(ii) comprises at least one Complementary Determining Region (CDR) selected from the group consisting of:
(a) 4B4 CDRH1 which has a sequence of SEQ ID NO: 21 or a sequence that differs from SEQ ID NO:21 by a single amino acid substitution;
(b) 4B4 CDRH2 which has a sequence of SEQ ID NO: 23 or a sequence that differs from SEQ ID NO:23 by a single amino acid substitution;
(c) 4B4 CDRH3 which has a sequence of SEQ ID NO: 25 or a sequence that differs from SEQ ID NO:25 by a single amino acid substitution;
(d) 4B4 CDRL1 which has a sequence of SEQ ID NO: 15 or a sequence that differs from SEQ ID NO:15 by a single amino acid substitution;
(e) 4B4 CDRL2 which has a sequence of SEQ ID NO: 17 or a sequence that differs from SEQ ID NO:17 by a single amino acid substitution; and
(f) 4B4 CDRL3 which has a sequence of SEQ ID NO: 19 or a sequence that differs from SEQ ID NO:19 by a single amino acid substitution; and/or
(iii) comprises a heavy chain variable region having a sequence at least 85%, 90%, 95%, 98%, 99%, or 100% identical to SEQ ID NO: 33; and/or
(iv) comprises a light chain variable region sequence having a sequence at least 85%, 90%, 95%, 98%, 99%, or 100% identical to SEQ ID NO: 31; or
(v) competes with the antibody of (i), (ii), (iii), or (iv).

20. The method or use of claim 18 or 19, or the first MCM5 binding agent and/or the second MCM5 binding agent for use according to claim 18 or 19, wherein:
(a) the first monoclonal antibody and/or second monoclonal antibody has an affinity for MCM5 in the range of 0.001-1 nM;
(b) the first monoclonal antibody and/or the second monoclonal antibody is a Fab′₂, a F(ab)₂, an Fv, a single chain antibody or a diabody;
(c) the first monoclonal antibody comprises 12A7 CDRH1, 12A7 CDRH2, and 12A7 CDRH3;
(d) the first monoclonal antibody comprises 12A7 CDRL1, 12A7 CDRL2 and 12A7 CDRL3;
(e) the first monoclonal antibody comprises a 12A7 CDRH1 which has a sequence of SEQ ID NO: 9, a 12A7 CDRH2 which has a sequence of SEQ ID NO: 11, and a 12A7 CDRH3 which has a sequence of SEQ ID NO: 13;
(f) the first monoclonal antibody comprises a 12A7 CDRL1 which has a sequence of SEQ ID NO: 3, a 12A7 CDRL2 which has a sequence of SEQ ID NO: 5, and a 12A7 CDRL3 which has a sequence of SEQ ID NO: 7;
(g) the second monoclonal antibody comprises 4B4 CDRH1, 4B4 CDRH2, and 4B4 CDRH3;
(h) the second monoclonal antibody comprises 4B4 CDRL1, 4B4 CDRL2 and 4B4 CDRL3;
(i) the second monoclonal antibody comprises a 4B4 CDRH1 which has a sequence of SEQ ID NO: 21, a 4B4 CDRH2 which has a sequence of SEQ ID NO: 23, and a 4B4 CDRH3 which has a sequence of SEQ ID NO: 25;
(j) the second monoclonal antibody has a 4B4 CDRL1 which has a sequence of SEQ ID NO: 15, a 4B4 CDRL2 which has a sequence of SEQ ID NO: 17, and a 4B4 CDRL3 which has a sequence of SEQ ID NO: 19;
(k) the first monoclonal antibody comprises a heavy chain variable region having a sequence at least 95% identical to SEQ ID NO: 29;
(l) the first monoclonal antibody comprises a heavy chain variable region having a sequence at least 98% identical to SEQ ID NO: 29;
(m) the first monoclonal antibody comprises a light chain variable region having a sequence at least 95% identical to SEQ ID NO: 27;
(n) the first monoclonal antibody comprises a light chain variable region having a sequence at least 98% identical to SEQ ID NO: 27;
(o) the second monoclonal antibody comprises a heavy chain variable region having a sequence at least 95% identical to SEQ ID NO: 33;
(p) the second monoclonal antibody comprises a heavy chain variable region having a sequence at least 98% identical to SEQ ID NO: 33;
(q) the second monoclonal antibody comprises a light chain variable region having a sequence at least 95% identical to SEQ ID NO: 31; and/or
(r) the second monoclonal antibody comprises a light chain variable region having a sequence at least 98% identical to SEQ ID NO: 31.

21. The method of any one of claims 1-7, 11-15, and 17-20, the use of any one of claims 8-9, 11-14, and 16-20, or the first MCM5 binding agent and/or the second MCM5 binding agent for use according to any one of claim 10-14 or 16-20, wherein the subject:
(i) is human;
(ii) is male;
(iii) is undergoing cystoscopic surveillance;
(iv) has been previously diagnosed as having bladder cancer;
(v) is in remission from a bladder cancer; and/or
(vi) is asymptomatic of a bladder cancer, optionally wherein the subject does not present with haematuria.

22. A kit comprising:
(i) a first MCM5 binding agent;
(ii) a second MCM5 binding agent; and/or
(iii) a lysis buffer capable of releasing MCM5 from cells in a urine sample; and
(iv) instructions for use of the first MCM5 binding agent, the second MCM5 binding agent, and/or the lysis buffer in: a method of detecting the presence or absence of a recurrent bladder cancer in a subject, a method for diagnosing a subject with a recurrent bladder cancer, and/or a method of monitoring a subject for recurrence of a bladder cancer.

23. The kit of claim 22, wherein:
the first MCM5 binding agent is as defined in any one of claim 10-14 or 16-20;
(ii) the second MCM5 binding agent is as defined in any one of claim 10-14 or 16-20;
(iii) the lysis buffer is as defined in any one of claims 5-7; and/or
(iv) the instructions are for use of the first MCM5 binding agent, the second MCM5 binding agent, and/or the lysis buffer in a method as defined in any one claims 1-7, 11-15, and 17-21.
